(19) United States
(12) Patent Application Publication
(10) Pub. No.: US 2015/0361403 A1
(43) Pub. Date: Dec. 17, 2015

(54) IMPROVED BACULOVIRAL EXPRESSION SYSTEM AND METHODS OF PRODUCING THE SAME

(71) Applicant: EUROPEAN MOLECULAR BIOLOGY LABORATORY, Heidelberg (DE)

(72) Inventors: Imre Berger, St. Egreve (FR); Deepak Balaji Thimiri Govinda Raj, Saint Martin le Vinoux (FR)

(21) Appl. No.: 14/763,740

(22) PCT Filed: Feb. 10, 2014
(86) PCT No.: PCT/IB2014/058885

§ 371 Date: Jul. 27, 2015

Related U.S. Application Data
(60) Provisional application No. 61/762,607, filed on Feb. 8, 2013.

(51) Int. Cl. C12N 7/00

(52) U.S. Cl. CPC C12N 7/00; C12N 2710/14121; C12N 2710/14151

(57) ABSTRACT

The present invention is based on the discovery that large parts of the genome of nucleopolyhedtovirus (NPV)-alpha baculovirus clade Ia viruses can be deleted with out deleterious effect on the usability of the virus comprising such genome in the infection of cells in cell culture. Accordingly, the present invention provides NPY-alpha baculovirus clade Ia genome which is reduced in size in comparison to the respective native NPV-alpha baculovirus clade Ia genome, such genomes comprising heterologous nucleotides, viruses comprising either of these genomes, cells infected with such viruses and methods for producing such viruses and cells.

### TECHNICAL FIELD OF THE INVENTION

The present invention is based on the discovery that large parts of the genome of nucleopolyhedrovirus (NPV)-alpha baculovirus clade Ia viruses can be deleted without deleterious effect on the usability of the virus comprising such genome in the infection of cells in cell culture. Accordingly, the present invention provides NPV-alpha baculovirus clade Ia genomes which are reduced in size in comparison to the respective native NPV-alpha baculovirus clade Ia genomes, such genomes comprising heterologous nucleotides, viruses comprising either of these genomes, cells infected with such viruses and methods for producing such viruses and cells.

### BACKGROUND OF THE INVENTION

The understanding of the cellular machinery has increased tremendously in recent years mainly due to astounding progress in ‘omics’ research (genomics, proteomics and glycomics) (Nie Y, et al (2009) Curr. Genomics 10:558-72). Comprehensive genomics datasets are now available for many organisms including human, and focus has now shifted to elucidating the cellular proteome in correlation with the live cellular functionality and morphology. One essential lesson learned is that proteins in eukaryotic cells typically do not work in isolation but coexist in large and highly diverse assemblies of ten or more interlocking subunits. These stably or transiently associated multiprotein assemblies additionally work together with separate proteins or multiprotein assemblies to carry out essential cellular processes including signaling, energy generation, and transport of food, water or waste.

A considerable number of accessory proteins typically accompany any individual multiprotein complex at various stages of its production, trafficking, active life and degradation. For example, chaperones are often critical for proper assembly of complexes, while other proteins are required for proper targeting and activation through post-translational modification. The activity of complexes is often fine-tuned by the incorporation of isoforms of individual subunits, for example to mediate tissue-specific functions. To fully understand biology, it is clear that new methods are needed to unlock the assembly, structure and mechanism of all of the complexes that exist in our cells. This is not only essential for basic research, but equally important for enabling novel approaches in the pharmaceutical and biotech industries to drive development of new and better drugs that more specifically modulate cellular functions. An imposing bottleneck that obstructs progress in these areas stems from the typically low abundance and high heterogeneity of protein complexes in their native cells. Apart from a handful of notable examples, most human multiprotein complexes remain virtually inaccessible to date.

Recombinant overexpression can provide a solution to this problem. However, until recently, the production challenge for eukaryotic (especially human) multiprotein complexes has not been systematically addressed. The provision of human multiprotein complexes in the quality and quantity required for mechanistic studies and drug design poses particular challenges due to the complexity of the machinery at work in our cells. Technical factors for heterologous protein production including protein yield, stoichiometric ratio between subunits, post-translational modifications, folding, and stability are all of critical importance, and ideally a highly flexible heterologous expression system should be available that can provide these functions for a wide range of protein complexes. An attractive solution could be mammalian expression systems, which naturally provide the required functions to accurately reflect what takes place in our cells, and heterologous expression in mammalian systems has become increasingly popular, especially for secreted proteins such as therapeutic antibodies (Nettleship J E, et al. (2010) J Struct. Biol. 172:55-65). However, mammalian systems often do not provide acceptable yields for intracellular proteins, and multiprotein expression technologies for mammalian cells are still in their infancy, albeit progress has been made recently, opening interesting options to depict with hitherto unattainable precision entire pathways in mammalian cells, for example for pharmacological screening studies (Kriz A, et al. (2010) Nat. Commun. 2010; 1:120).

An attractive alternative to mammalian systems is heterologous expression using recombinant baculoviruses to infect insect cell cultures. This method was pioneered three decades ago (Summers M D (2006) Adv. Virus Res. 68:3-73) and has become a method of choice for producing high levels of many eukaryotic proteins including a large number of proteins of pharmaceutical interest (Kost T A, et al. (2005) Nat. Biotechnol. 23:567-75 and Jarvis D L (2009) Methods Enzymol. 463:191-222). A significant advance over existing baculovirus expression vector systems (BEVS) came with the introduction of MultiBac, an advanced BEVS particularly tailored for producing eukaryotic multiprotein complexes for structural and functional studies (Bieniossek C et al. (2012) Trends Biochem. Sci. 37:49-57; Bieniossek C, et al. (2009) Nat Methods 6:447-50; Bieniossek C, et al. (2008) Curr. Protoc. Protein Sci. Chapter 5: Unit 5.20 and Fitzgerald D J, et al. (2006) Nat. Methods 3:1021-32). MultiBac consists of a baculovirus genome that has been engineered for optimized protein production by deleting protease and apoptosis activities (Barger I, et al. (2004) Nat. Biotechnol. 22:1583-7). In a subsequent improvement of the system, a new suite of transfer vectors was introduced to facilitate introduction of many heterologous genes into one recombinant MultiBac baculovirus by a method called Tandem Recombineering (TR), involving sequence-and-ligation-independent cloning (SLIC) and Cre-LoxP recombination (Trowitzsch S, et al. (2010) J. Struct. Biol. 172:45-5414; Vijayachandran L S, et al. (2011) J. Struct. Biol. 2011; 175:198-208). More recently, the design of transfer plasmids has been further refined, resulting in small, easy to handle plasmids containing only the functional DNA elements required for protein expression, expression cassette multiplication and plasmid concatenation by TR (Vijayachandran L S, et al. (2011) J. Struct. Biol. 175:198-208). Multigene transfer vectors created in this way are introduced into the MultiBac baculovirus genome by the Tn7 transposon, in E. coli strains modified for this purpose (Trowitzsch S, et al. (2010) supra).

As a step forward relative to previous systems, the original MultiBac system already provided the option to integrate accessory functionalities that may be required for proper functioning of a multiprotein complex, by means of a second entry site engineered into the virus genome that is independent of, and distal to the main site of integration that relies on the Tn7 transposition. This feature has been exploited to integrate additional functional modules into the viral genome, including post translational modification enzymes, and fluorescent proteins that allow easy monitoring of virus performance and protein production following transfection and during virus amplification (Vijayachandran L S, et al. (2011) and Fitzgerald D J, et al. (2007) Structure 15:275-9). More recently, this approach has been used to create SweetBac, allowing for the production of mammalian-like glycoproteins in insect cells (Palmberger D et al. (2012) PloS One 7:e34226 and Palmberger D et al. (2012) Bioengineered 2012; 4). MultiBac is now in use at more than 600 laboratories world-wide, in academia and industry, and a broad range of multiprotein complexes have been produced in high quality and quantity for diverse applications by using the MultiBac system (Trowitzsch S et al. (2012) supra; Nettleship J E et al. (2010) supra; Kriz A et al. (2010) supra; Summers M D (2005) supra; Jarvis D L (2009) Methods Enzymol. 463:191-222 and Bieniossek C (2012) Trends Biochem. Sci. 37:49-57).

Currently, two approaches for integrating heterologous expression cassettes into the baculovirus genome dominate the field. One of these approaches requires the presence of the baculoviral genome as a bacterial artificial chromosome (BAC) in E. coli cells, together with Tn7 transposase activity present in these same cells which recombine transformed transfer plasmids into a Tn7 attachment site on the BAC. Invitrogen's Bac-to-Bac system and also the more advanced MultiBac system both utilize this approach. The recombinant, composite baculovirus DNA is then purified from these E. coli cells by alkaline lysis, and used to transfect insect cells. In contrast, the original method of choice to integrate heterologous expression cassettes into the baculovirus genome relied on homologous recombination mediated by regions in the transfer plasmid that were homologous to two genes on the baculovirus genome (Orf1629 and lef2/603) that flank the baculoviral polh locus which had been inactivated. This method is still offered by a large number of commercial providers (Novagen BacVector series, Pharmingen BaculoGold, Abvector, others). By this method, homologous recombination occurs in insect cells following transfection the baculovirus genomic DNA together with the transfer vector. The efficiency of recombination is increased by linearization of the baculovirus genome, but still remains a less efficient method to rapidly generate recombinant baculovirus than transforming Tn7-produced composite BACs. A further improvement on the homologous recombination in insect cell method came by truncation of the essential Orf1629 gene on the baculovirus genome which is then repaired by co-transfecting complete Orf1629-containing transfer vectors (FlashBac system, Oxford Expresion Technologies, UK).

Currently, BEVS applications including MultiBac rely on a large baculovirus genome (130 kb) derived from wild-type Autographa californica multicapsid nuclear polyhedrosis virus (AcMNPV). This genome has been intensively researched for many years. Genes that are essential for propagation in cell culture and genes which are detrimental for foreign protein production were delineated by several research groups (Harrison R L t al. (2003) J. Gen. Virol. 2003; 84:1827-42; Pijlman G P et al. (2001) Virology 283:132-8; Pijlman G P et al. (2002) J. Virol. 76:5605-11; Pijlman G P t al. (2003) J. Gen. Virol. 84:2041-9; Pijlman G P et al. (2006), J. Biotechnol. 123:13-21; Pijlman G P at al. (2003) J. Gen. Virol. 2003; 84:2669-78 and Pijlman G P t al. (2003) J. Invertebr. Pathol. 84:214-9).

The inherent DNA instability of the currently used baculovirus genome poses a problem, in particular at expression scales relevant for pharmaceutical production. Simply speaking, as the virus replicates during expression scale up, it progressively suffers from deletion of bits and pieces of its genome, preferentially in the highly expressed, (non-essential) heterologous protein expression cassette, as was shown already for laboratory scale production (Fitzgerald D J (2006) Nat. Methods 3:1021-32; Pijlman G P (2001) Virology 283:132-8; Pijlman G P et al. (2002), J. Virol. 76:5605-11). This is exacerbated for the viruses of the BAC/Tn7 type by the fact that the insertion site which is targeted by the Tn7 transposon is actually a mutational hotspot (Carstens E B t al. (1987) J. Gen. Virol. 68:901-5; Roelvink P W et al. (1992) J. Gee. Virol. 73 (Pt 6):1481-9).

Accordingly, there is a need in the art to provide expression systems which allow efficient protein expression, accommodate large heterologous nucleotide inserts, e.g. to express multiple proteins and which are less prone to rearrangement of the genome, i.e. with improved genomic stability.

### SUMMARY OF THE INVENTION

In a first aspect the present invention relates to a nucleopolyhedrovirus (NPV)-alpha baculovirus clade Ia genome, wherein the number of base pairs is reduced in comparison to a native NPV-alpha baculovirus clade Ia genome by more than 18%, preferably an Autographa californica multicapsid nucleopolyhedrovirus (AcMNPV) genome, wherein the number of base pairs of the genome is reduced in comparison to a native BmNPV genome by at least at least 25.7% or a Bombyx mori nucleopolyhedrovirus (BmNPV) genome, wherein the number of base pairs of the genome is reduced in comparison to a native BmNPV genome by at least 18.31% and which in each case assembles into an infectious baculovirus.

In a second aspect the present invention relates to a NPV alpha baculovirus clade Ia genome according to the first aspect further comprising a nucleotide sequence heterologous to the NPV alpha baculovirus clade Ia genome.

In a third aspect the present invention relates to an infectious NPV alpha baculovirus clade Ia virus comprising a genome according to the first or second asp t of the invention.

In a fourth aspect the present invention relates to a cell infected with a virus according to the third aspect of the invention.

In a fifth aspect the present invention relates to a method for producing an NPV alpha baculovirus clade Ia genome according to the first or second aspect of the invention comprising the step of chemically synthesizing all or part of the genome.

In a sixth aspect the present invention relates to a method for producing an NPV alpha baculovirus clade Ia virus by introducing a genome according to the first or second aspect of the invention or producible according to the method of the fifth aspect into a cell.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### DEFINITIONS

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{th} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

The terms “polynucleotide” and “nucleic acid” are used interchangeably herein and are understood as a polymeric or oligomeric macromolecule made from nucleotide monomers.

Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA), deoxyribonucleic acid (DNA), and mixtures thereof such as e.g. RNA-DNA hybrids. The nucleic acids, can e.g. be synthesized chemically, e.g. in accordance with the phosphotriester method (see, for example, Uhlmann, E. & Peyman, A. (1990) Chemical Reviews, 90, 543-584). “Aptamers” are nucleic acids which bind with high affinity to a polypeptide. Aptamers can be isolated by selection methods such as SELEmir146-a (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability.

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitops and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility.

The term “sequence identity” is used throughout the specification with regard to polypeptide and polynucleotide sequence comparisons. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise. For example, a polypeptide sequence consisting of 200 amino acids compared to a reference 300 amino acid long polypeptide sequence may exhibit a maximum percentage of sequence identity of 66.6% (200/300) while a sequence with a length of 150 amino acids may exhibit a maximum percentage of sequence identity of 50% (150/300). If 15 out of those 150 amino acids are different from the respective amino acids of the 300 amino acid long reference sequence, the level of sequence identity decreases to 45%. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on httpJ/www.ebi.ac.uk/Tools/clustalw2/index.html or on http://npsa-pbil.ibcp.fr/cgi-bin/npsa_automat.pl?page=/NPSA/npsa_clustalw.htmi. Preferred parameters used are the default parameters as they are set on http://www.cbi.ac.uktTools/clustalw/or http://www.ebi.ac.ukfTools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. BLAST polynucleotide searches are performed with the BLASTN program, score=100, word length=12. BLAST protein searches are performed with the BLASTP program, score=50, word length=3. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise. “Hybridization” can also be used as a measure of sequence identity or homology between two nucleic acid sequences. A nucleic acid sequence encoding F, N, or M2-1, or a portion of any of these can be used as a hybridization probe according to standard hybridization techniques. Hybridization conditions are known to those skilled in the art and can be found, for example, in Current Protocols in Molecular Biology, John Wiley & Sons, N. Y., 6.3.1-6.3.6, 1991. “Moderate hybridization conditions” are defined as equivalent to hybridization in 2× sodium chloride/sodium citrate (SSC) at 30° C., followed by a wash in 1×SSC, 0.1% SDS at 50° C. “Highly stringent conditions” are defined as equivalent to hybridization in 6× sodium chloride/sodium citrate (SSC) at 45° C., followed by a wash in 0.2×SSC, 0.1% SDS at 65° C.

As used herein, the terms “resistance gene” refers to a gene conferring resistance to a toxin and/or an antibiotic”. Accordingly, such a gene may also be referred to as “toxin-resistance gene” or “antibiotica-resistance gene”. The functional inactivation of a toxin or antibiotic may be achieved by expressing a marker gene which carries mutation(s) rendering the respective gene product insensitive to a toxin or antibiotic. Alternatively, the functional inactivation of a toxin or antibiotic may be achieved by expressing a marker gene which inhibits the toxin or antibiotic e.g. by interacting or binding to it. The functional inactivation of a toxin or antibiotic may also be achieved by expressing a marker gene which counteracts the effects of the toxin or antibiotic.

Antibiotic compounds include but are not limited to tetracyclines, sulfonamides, penicillins, cephalosporins, ansamycins, carbapenems, macrolides, quinolones, aminonucleoside, aminoglycosides, peptides, glycopeptides, and lipopeptides. Exemplified, hygromycin B, neomycin, kanamycin, gentamicin, and G418 (also known as Geneticin) are aminoglycoside antibiotics which are similar in structure. In general, neomycin and kanamycin are used for prokaryotes, whilst G418 is needed for eukaryotes. Kanamycin is isolated from Streptomyces kanamyceticus and interacts with the 30S subunit of prokaryotic ribosomes thereby inducing mistranslation and indirectly inhibiting translocation during protein synthesis. Neomycin is produced naturally by the bacterium Streptomyces fradiae whilst G418 is produced by Micromonospora rhodoranges. Neomycin blocks protein biosynthesis by binding to the 30S subunit of the 70S-ribosome. G418 blocks polypeptide synthesis by binding to the 80S-ribosome and thereby inhibiting the elongation step in both prokaryotic and cukaryotic cells. Resistance to neomycin and G418 is conferred by the Neor gene from transposon Tn5 encoding an aminoglycoside 3′-phosphotransferase, APH 3′ II which phosphorylates neomycin or geneticin on a hydroxygroup and thereby, inhibits its function. Hygromycin B is produced by the bacterium Streptomyces hygroscopicus and kills bacteria, fungi and higher eukaryotic cells by inhibiting protein synthesis. The hygromycin resistance gene Hph encodes the hygromycin B phosphotransferase which inactivates hygromycin B through phosphorylation. Blasticidin is an antibiotic that is produced by Streptomyces griseochromogenes and prevents the growth of both eukaryotic and prokaryotic cells by inhibiting peptide bond formation by the ribosome. The three genes bis from Streptoverticillum sp., bsr from Bacillus cereus, and BSD from Aspergillus terreus, confer resistance to blasticidin by enabling the cells continue protein production even in the presence of blasticidin. Puromycin is an aminonucleoside antibiotic, derived from the Streptomyces alboniger that causes premature chain termination during translation taking place in the ribosome. The expression of the report gene Puror encodes a puromycin N-acetyl-transferase which conveys resistance to the antibiotic puromycin. In the context of the present invention genes conveying antibiotic-resistance are particularly suitable, including but not limited to genes conveying resistance to neomycin, puromycin, blasticidin and hygromycin.

### Embodiments

To provide improved expression systems the present inventors decided to rewire the entire baculovirus genome to maximize its performance. The aim was the redesign and restructuring of the baculovirus genome to provide among other advantages enhanced DNA stability and efficient protein production. The genes and DNA elements which are dispensable under laboratory culture conditions and unnecessary for efficient budded viral production, which is the major virus type used for protein expression in cell culture (Bieniossek C et al. (2008) supra and Fitzgerald D J et al (2006) Nat. Methods 3:1021-32) have been determined by the present inventors. This allowed engineering an improved baculovirus genome by removing non-essential genes and regions prone to mutation. Such engineered viruses provide among other advantages improved virus DNA stability, increased ability of accommodating very large foreign gene insertions, without compromising the ease of handling and superior protein production properties.

The present inventors surprisingly found that the size of the genome of a NPV-alpha baculovirus clade Ia genome can be significantly reduced without affecting its ability to infect and propagate in cells. Viruses comprising such an optimized genome provide enhanced DNA stability and more efficient protein production. Thus, in a first aspect this invention provides a genome of a nucleopolyhedrovirus (NPV)-alpha baculovirus clade Ia virus, wherein the number of base pairs is reduced in comparison to a native NPV-alpha baculovirus clade Ia genome by more than 18% and which assembles into an infectious baculovirus, e.g. under the conditions outlined below, preferably a genome of an Autographa californica multicapsid nucleopolyhedrovirus (AcMNPV), wherein the number of base pairs of the genome is reduced in comparison to a native AcMNPV genome by at least 25.7% or a Bombyx mori nucleopolyhedrovirus (BmNPV) genome, wherein the number of base pairs of the genome is reduced in comparison to a native BmNPV genome by at least 18.31% and which in each case assembles into an infectious baculovirus.

Preferably, the genome assembles into an infectious baculovirus capable of expressing heterologous proteins, once it infects a permissible cell.

The genomes of baculoviruses belonging to the group of NPV alpha baculovirus clade Ia are highly conserved. Thus, in the following the coding segments (CDS), 5′ untranslated regions (UTRs), spacers and/or repeat sequences (br) to be deleted or maintained are always indicated with reference to the genome of AcMNPV or BmNPV. If nucleotide positions are indicated these are either with reference to the genome of AcMNPV according to SEQ ID NO: 1 or of BmNPV according to SEQ ID NO: 4. Based on this information the skilled person can determine without undue burden the corresponding CDS, UTR, spacer or hr sequence of another NPV alpha baculovirus clade Ia virus by using standard alignment tools as set out above. The result of such an approach is exemplary described for the ptp gene encoding a protein tyrosine phosphatase of AcMNPV (UniProt P24656, SEQ ID NO: 13). When the amino acid sequence of SEQ ID NO: 13 is used in a PBLAST search of non-redundant protein sequences the homologs of the protein tyrosine phosphatase of AcMNPV from other NPV alpha baculovirus clade Ia viruses are identified, e.g. a homolog from Rachiplusia ou multiple nucleopolyhedrovirus (RoMNPV) (Accession NO: NP_{—}702993; SEQ ID NO: 14), a homolog from BmNPV (Accession No: AAG31657; SEQ ID NO: 15), a homolog from Maruca vitrata multiple nuclopolyhcdrovirus (MaviMNPV) (Accession No.: YP_{—}950853; SEQ ID NO: 16), a homolog from Choristoneura fumiferana defective nucleopolyhedroviru (CfDefMNPV) (Accession No.: NP_{—}932617; SEQ ID NO: 17), a homolog of Anticarsia gemmatalis multiple nucleopolyhedrovirus (AgMNPV) (Accession No.: YP_{—}803403; SEQ ID NO: 18), a homolog from Epiphyas postvittana nucleopolyhedrovirus (EppoNPV) (Accession No: NP_{—}203176; SEQ ID NO: 19), a homolog from Antheraea pernyi nucleopolyhedrovirus (AnpeNPV) (Accession No.: YP_{—}611104; SEQ ID NO: 20), a homolog from Choristoneura Fumiferana Multinucleocapsid nucleopolyhedrovirus (CfMNPV) (Accession No.: NP_{—}848321; SEQ ID NO: 21), a homolog from Orgyia Pseudotsugata Multicapsid nucleopolyhedrovirus (OpMNPV) (Accession No.: NP_{—}046166; SEQ ID NO: 22), and a bomolog from Hyphantria cunea nucleopolyhedrovirus (HycuNPV) (Accession No: YP_{—}473330; SEQ ID NO: 23). The alignment of these sequences using CLUSTALW2 is shown in FIG. 2. It is clear that the ptp protein is highly conserved among different NPV alpha baculovirus clade Ia viruses. Accordingly, if the ptp gene is deleted in a NPV alpha baculovirus clade Ia virus this means that at least the CDS encoding the ptp protein in the respective NPV alpha baculovirus clade Ia virus is deleted. It is preferred that also the 5′-UTR of the respective CDS is deleted. In the following reference is always made to genes encoding specific NPV alpha baculovirus clade Ia virus proteins. To unambiguously identify the respective gene the UniProt reference number of the protein of AcMNPV or BmNPV is indicated. Nevertheless, the homologs of that gene in other NPV alpha baculovirus clade Ia virus are also referred to. Similarly, the 5′ and 3′ end of a CDS, 5′-UTR, hr or spacer are always indicated with reference to the AcMNPV genome according to SEQ ID NO: 1 or with reference to the genome of BmNPV, however, include the homologous regions from other NPV alpha baculovirus clade Ia virus, which can be identified by the skilled person without undue burden by alignment of the referenced CDS, 5′UTRs, hrs and spacers, respectively, of (i) the AcMNPV genome according to SEQ ID NO: 1, (ii) the BmNPV genome according to SEQ ID NO: 4 or (iii) the AcMNPV genome according to SEQ ID NO: 1 and the BmNPV genome according to SEQ ID NO: 4 with the genome of the other NPV alpha baculovirus clade Ia virus. While the NPV alpha baculoviruses are highly conserved amongst each other, it is possible that for a given AcMNPV or BmNPV CDS, 5′-UTR, hr and/or spacer region to be deleted no homologous region can be identified in the baculovirus to be modified. This may be due to the fact that the particular NPV clade Ia virus may have lost this genomic segment naturally and, thus, that it will not be possible to delete a genomic segment homologous to the reference segment of AcMNPV and/or BmNPV. In the determination of homologous segments it is preferred that the reference nucleotide sequence to be deleted or maintained and the nucleotide sequence from another clade Ia baculovirus show an identity of at least 60%, 65%, 70%, 75, 80%, 85%, 90%, 95% or even 100%. If a homologous segment for a CDS is determined it is preferred that the sequence comparison is carried out on the basis of the encoded amino acid rather than on the basis of the coding sequence. It is then preferred that a homologous protein shows an amino acid identity of at least 70%, 75, 80%, 85%, 90%_{,} 95% or even 100% with the reference amino acid sequence of the AcMNPV or BmNPV protein.

Preferably the size of the NPV alpha baculovirus clade Ia genome is reduced by at least 20%, more preferably by at least 22%, at least 24%, at least 26%, at least 28% at least 30% at least 32%, at least 34%, at least 36% at least 38%, or at least 40% % by following the teaching in this application, which genes to maintain and to delete with respect to the native genome of the respective virus to arrive at the genome with a reduced size of the present invention.

Preferably the size of genome of AcMNPV is reduced by at least 26%, at least 28% at least 30% at least 32%, at least 34%, at least 36% at least 38%, at least 40%, Preferably the size of genome of BmNPV is reduced by at least 20%, more preferably by at least 22%, at least 24%, at least 26%, at least 28% at least 30% at least 32%, at least 34%, at least 36% at least 38%, or at least 40%.

Preferably the size of genome of RoMNPV is reduced by at least 20%, more preferably by at least 22%, at least 24%, at least 26%, at least 28% at least 30% at least 32%, at least 34%, at least 36% at least 38%, or at least 40%.

Preferably the size of genome of MaviMNPV is reduced by at least 20&, more preferably by at least 22%, at least 24%, at least 26%, at least 28% at least 30% at least 32%, at least 34%, at least 36% at least 38%, or at least 40%.

Preferably the size of genome of CfDefMNPV is reduced by at least 20%, more preferably by at least 22%, at least 24%, at least 26%, at least 28% at least 30% at least 32%, at least 34%, at least 36% at least 38%, or at least 40%.

Preferably the size of genome of AgMNPV is reduced by at least 20%, more preferably by at least 22%, at least 24%, at least 26%, at least 28% at least 30% at least 32%, at least 34%, at least 36% at least 38%, or at least 40%.

Preferably the size of genome of EppoNPV is reduced by at least 20%, more preferably by at least 22%, at least 24%, at least 26%, at least 28% at least 30% at least 32%, at least 34%, at least 36% at least 38%, or at least 40%.

Preferably the size of genome of AnpeNPV is reduced by at least 20%, more preferably by at least 22%, at least 24%, at least 26%, at least 28% at least 30% at least 32%, at least 34%, at least 36% at least 38%, or at least 40%.

Preferably the size of genome of CfMNPV is reduced by at least 20%, more preferably by at least 22%, at least 24%, at least 26%, at least 28% at least 30% at least 32%, at least 34%, at least 36% at least 38%, or at least 40%.

Preferably the size of genome of OpMNPV is reduced by at least 20% o, more preferably by at least 22%, at least 24%, at least 26%, at least 28%, at least 30% at least 32%, at least 34%, at least 36% at least 38%, or at least 40%.

Preferably the size of genome of HycuNPV is reduced by at least 20%, more preferably by at least 22%, at least 24%, at least 26%, at least 28% at least 30% at least 32%, at least 34%, at least 36% at least 38%, or at least 40%.

Preferably the size of genome of Plutella xylostella nucleopolyhedrovirus (PlxyNPV) is reduced by at least 20%, more preferably by at least 22%, at least 24%, at least 26%, at least 28% at least 30%, at least 32%, at least 34%, at least 36% at least 38%, or at least 40%.

In above and below part of the description the reduction in size is always expressed as a percentage of the reduction in comparison to a native baculovirus genome. Alternatively the reduction in size may also be indicated in absolute values, i.e. reduction in size by X base pairs. The length of the CDS, 5′-UTR, spacer and hr segments, respectively, in base pairs are indicated in detail below in Tables 1 to 12 for AcMNPV and BmNPV (see column labeled “bp” in each Table). The sum of the bp of the respectively indicated elements will determine the number of base pairs that the baculovirus genome is shortened with respect to the native baculovirus genome, preferably those according to SEQ ID NO: 1 or 15. Using the teaching on how to identify homologous regions in other Clade Ia viruses by sequence alignment the skilled person can also determine the length of the homologous segments in other Clade Ia viruses and add the number of base pairs of the homologous sequences to be deleted to arrive at the value for absolute reduction of the length of the native genome of that particular Clade Ia baculovirus.

The present inventors have found that the CDS/proteins indicated below can be deleted without detrimeantal effect on the respective NPV alpha baculovirus clade Ia virus (all CDS and proteins are with reference to the genome of AcMNPV and BmNPV, respectively).

The present inventors found that the CDS indicated below in Tables 1a can be deleted without detrimental effect on AcMNPV. Further, the CDS indicated below in Tables 1b can be deleted without detrimental effect on BmNPV. These CDS are considered to belong to a group that is referred to as Type I of the respective NPV alpha baculovirus clade Ia virus (all CDS and proteins are with reference to AcMNPV). Accordingly, the deletion of these CDS (and preferably also the respective 5′-UTR, spacer and/or hr regions) are referred to as Type I deletions:

TABLE 1a
AcMNPV

| Name | UniProt | Definition | aa | bp | Start | Stop | Strand |
|---|---|---|---|---|---|---|---|
| Ptp | P24656 | protein tyrosine phosphatase | 168 | 507 | 503 | 1009 | plus |
| Bro | P24655 | baculovirus repeated ORF | 328 | 987 | 1041 | 2027 | minus |
| Ctx | P41416 | conotoxin-like peptide | 53 | 162 | 2084 | 2245 | minus |
| orf603 | P24650 | hypothetical protein ACNVgp007 | 201 | 606 | 3759 | 4364 | minus |
| polyhedrin | P04871 | major occlusion body protein | 245 | 738 | 4520 | 5257 | plus |
| Egt | P18569 | ecdysteroid UDP-glucosyl | 506 | 1521 | 11426 | 12946 | plus |
| | | transferase | | | | | |
| bv/odv-e26 | P12827 | AcOrf-16 peptide | 225 | 678 | 13092 | 13769 | plus |
| ac18 | P12828 | AcOrf-18 peptide | 353 | 1062 | 14398 | 15459 | minus |
| pif-2 | P41427 | AcOrf-22 peptide | 382 | 1149 | 17301 | 18449 | plus |
| env-prot | P41428 | copia-like envelope protein | 690 | 2073 | 18513 | 20585 | plus |
| iap-1 | P41435 | apoptosis inhibitor | 286 | 861 | 22600 | 23460 | plus |
| Sod | P24705 | superoxide dismutase | 151 | 456 | 25820 | 26275 | plus |
| Fgf | P41444 | fibroblast growth factor | 181 | 546 | 27041 | 27586 | minus |
| v-ubi | P16709 | viral ubiquitin | 77 | 234 | 28962 | 29195 | plus |
| p43 | P34050 | hypothetical protein ACNVgp039 | 363 | 1092 | 31078 | 32169 | minus |
| odv-e66 | Q00704 | occlusion-derived virus envelope | 704 | 2115 | 36718 | 38832 | plus |
| | | protein | | | | | |
| gp37 | P23058 | fusolin; spindle body protein; | 302 | 909 | 51283 | 52191 | minus |
| | | 34.8K; SLP; spheroidin-like | | | | | |
| | | protein; p34.8 | | | | | |
| odv-nc42 | P41468 | Virus structure | 192 | 579 | 58720 | 59298 | plus |
| ac69 | P41469 | putative methyl transferase | 262 | 789 | 59276 | 60064 | plus |
| iap-2 | P41454 | apoptosis inhibitor | 249 | 750 | 61016 | 61765 | plus |
| pnk/pnl | P41476 | polynucleotide kinase/ligase; | 694 | 2085 | 72131 | 74215 | minus |
| | | pnk | | | | | |
| ac91 | P41479 | AcOrf-91 peptide | 224 | 675 | 77987 | 78661 | minus |
| odv-e28 | P41656 | pif-4 AcOrf-96 peptide | 173 | 522 | 84346 | 84867 | plus |
| pif-4 | | | | | | | |
| pif-3 | P41668 | AcOrf-115 peptide | 204 | 615 | 99182 | 99796 | minus |
| pif-1 | P41672 | AcOrf-119 peptide | 530 | 1593 | 100699 | 102291 | plus |
| pk-2 | P41676 | protein kinase, GCN2-like kinase | 215 | 648 | 102964 | 103611 | minus |
| chiA | P41684 | chitinase | 551 | 1656 | 105282 | 106937 | minus |
| v-cath | P25783 | viral cathepsin-like protein | 323 | 972 | 106983 | 107954 | plus |
| pp34 | P24728 | major polyhedral calyx protein | 252 | 759 | 110903 | 111661 | plus |
| 94K | P08161 | hypothetical protein ACNVgp135 | 803 | 2412 | 113870 | 116281 | minus |
| p26 | P08358 | hypothetical protein ACNVgp137 | 240 | 723 | 118044 | 118766 | plus |
| p10 | P04870 | fibrous body protein | 94 | 285 | 118839 | 119123 | plus |
| p74 | P15963 | occlusion-derived virus envelope | 645 | 1938 | 119135 | 121072 | minus |
| | | protein, pif | | | | | |
| ac145 | P41703 | AcOrf-145 peptide | 77 | 234 | 126299 | 126532 | plus |
| odv-e56 | P41705 | occlusion-derived virus envelope | 376 | 1131 | 129008 | 130138 | minus |
| | | protein | | | | | |
| ac150 | P41707 | AcOrf-150 peptide | 99 | 300 | 130456 | 130755 | plus |

TABLE 1b
BmNPV

| Name | UniProt | Definition | aa | bp | Start | Stop | Strand |
|---|---|---|---|---|---|---|---|
| Polyhedrin | 1724487 | Polyhedrin | 245 | 738 | 1 | 738 | plus |
| Egt | 1488637 | UDP-l | 506 | 1521 | 6407 | 7927 | plus |
| | | Glucosy | | | | | |
| | | Transferase | | | | | |
| bv/odv-e26 | 1488639 | BV/ODV- | 229 | 690 | 8067 | 8756 | plus |
| | | E26 | | | | | |
| bm10 ac18 | 1488641 | AcMNPV | 356 | 1071 | 9387 | 10457 | minus |
| | | orf18 | | | | | |
| pif-2 | 1488644 | AcMNPV | 382 | 1149 | 12335 | 13483 | plus |
| | | orf22 | | | | | |
| env-prot | 1488645 | AcMNPV | 673 | 2022 | 13586 | 16607 | plus |
| | | orf23 | | | | | |
| iap-1 | 1488649 | IAP1 | 292 | 879 | 17606 | 18484 | plus |
| Fgf | 1488656 | FGF | 182 | 549 | 23407 | 23955 | plus |
| v-ubi | 1724483 | Ubiquitin | 77 | 234 | 25035 | 25268 | plus |
| bm57 ac69 | 1488688 | AcMNPV | 262 | 789 | 54440 | 55228 | plus |
| | | orf69 | | | | | |
| bm74 ac91 | 1488706 | AcMNPV | 154 | 465 | 69985 | 70449 | minus |
| | | orf91 | | | | | |
| pif-3 | 1488726 | AcMNPV | 204 | 615 | 91385 | 91999 | minus |
| | | orf115 | | | | | |
| pif-1 | 1488729 | AcMNPV | 527 | 1584 | 92532 | 94115 | plus |
| | | orf119 | | | | | |
| chi-a | 1724489 | CHITINASE | 552 | 1659 | 97049 | 98707 | minus |
| v-cath | 1724490 | Cystein | 323 | 972 | 98756 | 99727 | plus |
| | | Protease | | | | | |
| pp34 | 1488739 | PP34 | 315 | 948 | 102560 | 103507 | plus |
| bm110a | 1488742 | P94 | 57 | 174 | 105505 | 105678 | minus |
| 94K ac134 | | | | | | | |
| p26 | 1488745 | P26 | 240 | 723 | 107702 | 108424 | plus |
| p10 | 1488746 | P10 | 70 | 213 | 108497 | 108709 | plus |
| p74 | 1488747 | P74 | 645 | 1938 | 108796 | 110733 | minus |
| bm121/ | 1488753 | AcMNPV | 95 | 288 | 116041 | 116328 | plus |
| ac145 | | orf145 | | | | | |
| bm126 | 1488758 | AcMNPV | 115 | 348 | 120282 | 120629 | plus |
| ac150 | | orf150 | | | | | |
| Ptp | 1488762 | PTP | 168 | 507 | 124431 | 124937 | plus |
| bro-d | 1488763 | BRO-d | 349 | 1050 | 124934 | 126983 | Minus |
| Gta | 1488664 | GTA | 506 | 1521 | 30152 | 31672 | plus |
| gp37 | 1488684 | GP37 | 294 | 885 | 46479 | 47363 | minus |

The total length of the CDS encoding these portions is approximately 34,362 base pairs in AcMNPV according to SEQ ID NO: 1. Thus, the native AcMNPV genome according to SEQ ID NO: 1 of a length of 133,894 base pairs is preferably shortened by deletion of the CDS encoding these proteins and, thus, the AcMNPV genome of the invention is preferably at least 34,363 base pairs shorter than the native AcMNPV genome. The total length of the CDS encoding these proteins is approximately 24,531 base pairs in BmNPV. Thus, the native BmNPV genome having a length of 128,413 base pairs is preferably shortened by deletion of the CDS encoding these proteins and, thus, the BmNPV genome of the invention is preferably at least 25,121 base pairs shorter than the native BmNPV genome.

Accordingly, the AcMNPV genome of the invention is at least 25.7% shorter than the native AcMNPV genome. Accordingly, the BmNPV genome of the invention is at least 18.31% shorter than the native BmNPV genome.

For MvMNPV, the genome of the invention is preferably at least 21,688 bases pairs shorter than the native MvMNPV genome. For BmaMNPV, the genome of the invention is preferably at least 20,235 bases pairs shorter than the native BmaMNPV genome. For BmNPV, the genome of the invention is preferably at least 24,531 bases pairs shorter than the native BmNPV genome. For PlxyMNPV, the genome of the invention is preferably at least 26,724 bases pairs shorter than the native PlxyMNPV genome. For RoMNPV, the genome of the invention is preferably at least 26,379 bases pairs shorter than the native RoMNPV genome.

It is more preferred that one or more of the 5′-UTRs of these CDS are also deleted. In AcMNPV the 5′-UTRs of these CDS have a length of 2,113 base pairs. In BmNPV the 5′-UTRs of these CDS have a length of 1,614 base pairs. The following Tables 2a and 2b indicate the position of the 5′UTRs of the CDS deleted in preferred genomes of the invention. The 5′-UTR precedes the respectively indicated CDS Start codon by the indicated number of base pairs:

TABLE 2a
AcMNPV

| Name | GeneID | Start | Stop | Strand | Bp |
|---|---|---|---|---|---|
| ptp | 1403833 | 503 | 1009 | Plus | 57 |
| bro | 1403834 | 1041 | 2027 | minus | 56 |
| ctx | 1403835 | 2084 | 2245 | minus | 49 |
| orf603 | 1403839 | 3759 | 4364 | minus | 155 |
| polyhedrin | 1403840 | 4520 | 5257 | Plus | 155 |
| egt | 1403847 | 11426 | 12946 | Plus | 112 |
| bv/odv-e26 | 1403848 | 13092 | 13769 | Plus | 145 |
| ac18 | 1403850 | 14398 | 15459 | minus | 1 |
| pif-2 | 1403854 | 17301 | 18449 | Plus | 36 |
| env-prot | 1403855 | 18513 | 20585 | Plus | 63 |
| iap-1 | 1403859 | 22600 | 23460 | Plus | 1 |
| sod | 1403863 | 25820 | 26275 | Plus | 113 |
| fgf | 1403864 | 27041 | 27586 | minus | 146 |
| v-ubi | 1403867 | 28962 | 29195 | Plus | 20 |
| p43 | 1403871 | 31078 | 32169 | minus | 7 |
| odv-e66 | 1403878 | 36718 | 38832 | Plus | −16 |
| gp37 | 1403897 | 51283 | 52191 | minus | 137 |
| odv-nc-42 | 1403901 | 58720 | 59298 | Plus | −159 |
| ac69 | 1403902 | 59276 | 60064 | Plus | −23 |
| iap-2 | 1403904 | 61016 | 61765 | Plus | 33 |
| pnk/pnl | 1403919 | 72131 | 74215 | minus | 140 |
| ac91 | 1403924 | 77987 | 78661 | minus | 37 |
| odv-e28 | 1403929 | 84346 | 84867 | Plus | −14 |
| pif-3 | 1403948 | 99182 | 99796 | minus | 7 |
| pif-1 | 1403952 | 100699 | 102291 | Plus | −6 |
| pk-2 | 1403956 | 102964 | 103611 | minus | 181 |
| chiA | 1403959 | 105282 | 106937 | minus | 45 |
| v-cath | 1403960 | 106983 | 107954 | Plus | 45 |
| pp34 | 1403964 | 110903 | 111661 | Plus | 58 |
| 94K | 1403967 | 113870 | 116281 | minus | 210 |
| p26 | 1403969 | 118044 | 118766 | Plus | 56 |
| p10 | 1403970 | 118839 | 119123 | Plus | 72 |
| p74 | 1403971 | 119135 | 121072 | minus | 132 |
| ac145 + 18 | 1403978 | 126299 | 126532 | Plus | 69 |
| odv-e56 | 1403981 | 129008 | 130138 | minus | 28 |
| ac150 | 1403983 | 130456 | 130755 | Plus | −35 |

TABLE 2b
BmNPV

| Name | GeneID | Start | Stop | Strand | Bp |
|---|---|---|---|---|---|
| polyhedrin | 1724487 | 1 | 738 | plus | 129 |
| egt | 1488637 | 6407 | 7927 | plus | 114 |
| bv/odv-e26 | 1488639 | 8067 | 8756 | plus | −35 |
| bm10 ac18 | 1488641 | 9387 | 10457 | minus | 1 |
| pif-2 | 1488644 | 12335 | 13483 | plus | 36 |
| env-prot | 1488645 | 13586 | 15607 | plus | 102 |
| iap-1 | 1488649 | 17606 | 18484 | plus | 1 |
| fgf | 1488656 | 23407 | 23955 | plus | 206 |
| v-ubi | 1724483 | 25035 | 25268 | plus | 20 |
| bm57 ac69 | 1488688 | 54440 | 55228 | plus | −23 |
| bm74 ac91 | 1488706 | 69985 | 70449 | minus | 35 |
| pif-3 | 1488726 | 91385 | 91999 | minus | 7 |
| pif-1 | 1488729 | 92532 | 94115 | minus | 131 |
| chi-a | 1724489 | 97049 | 98707 | minus | 48 |
| v-cath | 1724490 | 98756 | 99727 | plus | 48 |
| pp34 | 1488739 | 102560 | 103507 | plus | 61 |
| bm110a | 1488742 | 105505 | 105678 | minus | −102 |
| 94K ac134 | | | | | |
| p26 | 1488745 | 107702 | 108424 | plus | 140 |
| p10 | 1488746 | 108497 | 108709 | plus | 72 |
| p74 | 1488747 | 108796 | 110733 | minus | 230 |
| bm121/ | 1488753 | 116041 | 116328 | plus | 14 |
| ac145 | | | | | |
| bm126 | 1488758 | 120282 | 120629 | plus | −32 |
| ac150 | | | | | |
| ptp | 1488762 | 124431 | 124937 | plus | 133 |
| bro-d | 1488763 | 124934 | 125983 | Minus | 74 |
| gta | 1488664 | 30152 | 31672 | plus | 75 |
| gp37 | 1488684 | 46479 | 47363 | minus | 129 |

The deletion of one or more of these 5′-UTRs from the genome of a NPV alpha baculovirus clade Ia virus leads to a further reduction of the size of the genome in comparison to the native genome of up to 1.58%. Accordingly, in an even more preferred embodiment the size of the genome of the AcMNPV virus of the invention is reduced by at least 27.24%. Accordingly, in an even more preferred embodiment the size of the genome of the BmNPV virus of the invention is reduced by at least 19.56%.

Alternatively or additionally the spacers of the AcMNPV CDS may be deleted, which amount to an additional deletion of 3,000 bp. In BmNPV the spacers of these CDS have a length of 3,047 base pairs. With reference to the nucleotide sequence of AcMNPV the spacers that may be deleted are the following:

TABLE 3a
AcMNPV

| Gene-Before | Gene-After | Start | Stop | bp |
|---|---|---|---|---|
| hr1 | ptp | 446 | 502 | 57 |
| ptp | bro | 1010 | 1040 | 31 |
| bro | ctx | 2028 | 2083 | 56 |
| ctx | Ac4 | 2246 | 2294 | 49 |
| lef2 | ORF603 | 3722 | 3758 | 37 |
| ORF603 | PH | 4365 | 4519 | 155 |
| PH | ORF1629 | 5258 | 5286 | 29 |
| lef1 | egt | 11314 | 11425 | 112 |
| egt | Ac16 | 12947 | 13091 | 145 |
| Ac17 | Ac18 | 14233 | 14397 | 165 |
| Ac18 | Ac19 | 15460 | 15460 | 1 |
| Ac22 | env-prot | 18450 | 18512 | 63 |
| env-prot | pkip | 20586 | 20633 | 48 |
| Ac26 | IAP1 | 22599 | 22599 | 1 |
| IAP1 | lef6 | 23461 | 23464 | 4 |
| Ac30 | sod | 25707 | 25819 | 113 |
| sod | hr2 | 26276 | 26292 | 17 |
| hr2 | fgf | 26962 | 27040 | 79 |
| fgf | HisP | 27587 | 27732 | 146 |
| Ac34 | v-ubi | 28942 | 28961 | 20 |
| v-ubi | 39K/pp31 | 29196 | 29241 | 46 |
| Ac38 | p43 | 31015 | 31077 | 63 |
| p43 | p47 | 32170 | 32176 | 7 |
| odv-e66 | Ac47 | 38833 | 38937 | 105 |
| Ac63 | gp37 | 51263 | 51282 | 20 |
| gp37 | DNA-pol | 52192 | 52328 | 137 |
| Ac69 | Ac70 | 60065 | 60109 | 45 |
| Ac70 | IAP2 | 60983 | 61015 | 33 |
| IAP2 | Ac72 | 61766 | 61823 | 58 |
| Ac85 | PNK/PNL | 72096 | 72130 | 35 |
| PNK/PNL | p15 | 74216 | 74355 | 140 |
| Ac91 | Ac92 | 78662 | 78698 | 37 |
| Ac122 | pk-2 | 102902 | 102963 | 62 |
| pk-2 | Ac124 | 103612 | 103792 | 181 |
| lef7 | chitinase | 105234 | 105281 | 48 |
| chitinase | v-cath | 106938 | 106982 | 45 |
| v-cath | gp64 | 107955 | 108178 | 224 |
| gp16 | PE/pp34 | 110845 | 110902 | 58 |
| PE/pp34 | Ac132 | 111662 | 111872 | 211 |
| 94K | 35K/p35 | 116282 | 116491 | 210 |
| hr5 | p26 | 117988 | 118043 | 56 |
| p26 | p10 | 118767 | 118838 | 72 |
| p10 | p74 | 119124 | 119134 | 11 |
| p74 | ME53 | 121073 | 121204 | 132 |
| odv-ec27 | Ac145 | 126230 | 126298 | 69 |
| IE-01 | odv-e56 | 128947 | 129007 | 61 |
| odv-e56 | Ac149 | 130139 | 130166 | 28 |
| Ac150 | IE-2 | 130756 | 130856 | 101 |

TABLE 3b
BmNPV

| Gene-After | Gene-before | Start | Stop | bp |
|---|---|---|---|---|
| Lef-2 | polyhedrin | 128285 | 128413 | 129 |
| Lef-1 | egt | 6293 | 6406 | 114 |
| bm9 ac17 | bm10 ac18 | 9358 | 9386 | 29 |
| bm17 ac26 | iap-1 | 17605 | 17605 | 1 |
| hr2L | fgf | 23201 | 23406 | 206 |
| bm25 ac34 | v-ubi | 25015 | 25034 | 20 |
| bm57 ac69 | iap2 | 55229 | 55376 | 148 |
| bm74 ac91 | bm75 ac92 | 70450 | 70484 | 35 |
| chi-a | v-cath | 98708 | 98755 | 48 |
| v-cath | gp64/67 | 99728 | 99843 | 116 |
| gp16 | pp34 | 102499 | 102559 | 61 |
| alk-exo | bm110a ac134 | 105463 | 105504 | 42 |
| hr5 | p26 | 107562 | 107701 | 140 |
| p26 | p10 | 108425 | 108496 | 72 |
| p10 | p74 | 108710 | 108795 | 86 |
| odv-ec27 | bm121 ac145 | 116027 | 116040 | 14 |
| bm126 | ie-2 | 120630 | 120661 | 32 |
| ac150 | | | | |
| hr1 | ptp | 124298 | 124430 | 133 |
| Bro-e | cds | 125984 | 126057 | 74 |
| bm32 ac41 | gta | 30077 | 30151 | 75 |
| bm51 ac63 | gp37 | 46403 | 46478 | 76 |
| ph | orf1629 | 739 | 767 | 29 |
| egt | bm7a | 7928 | 7939 | 12 |
| bm10 ac18 | bm11 ac19 | 10458 | 10458 | 1 |
| iap-1 | lef-6 | 18485 | 18488 | 4 |
| fgf | hr2R | 23956 | 24011 | 56 |
| v-ubi | 39k | 25269 | 25320 | 52 |
| iap2 | bm58a ac72 | 56127 | 56184 | 58 |
| v-cath | gp64/67 | 99728 | 99843 | 116 |
| gp64/67 | p24 | 101437 | 101562 | 126 |
| pp34 | bm109 ac132 | 103508 | 103509 | 2 |
| p26 | p10 | 118767 | 118838 | 72 |
| p10 | p74 | 119124 | 119134 | 11 |
| p74 | me53 | 110734 | 110963 | 230 |
| ie-2 | pe38 | 121931 | 122415 | 485 |
| gta | bm34 ac43 | 31673 | 31685 | 13 |
| gp37 | dna pol | 47364 | 47492 | 129 |

The deletion of these spacers from the genome of a NPV alpha baculovirus clade Ia virus leads to a further reduction of the size of the genome in comparison to the native genome. Specifically, the deletion of these spacers from the genome of a AcMNPV virus leads to a further reduction of the size of the genome in comparison to the native genome of 2.24%. Accordingly, in an even more preferred embodiment the size of the genome of the AcMNPV virus of the invention is reduced by at least 29.48%.

The deletion of these spacers from the genome of a BmNPV virus leads to a further reduction of the size of the genome in comparison to the native genome of 2.33%. Accordingly, in an even more preferred embodiment the size of the genome of the BmNPV virus of the invention is reduced by at least 20.36%.

It is noted that some of the spacers indicated above overlap with the 5′UTRs indicated in Table 2a or 2b. Thus, if the 5′-UTR is deleted for a given gene and the spacer is deleted additionally this means that only that part of the spacer that is not overlapping with a 5′-UTR is deleted. Conversely, if a spacer is deleted that partially overlaps with a 5′-UTR the overlapping part is also deleted.

It is preferred that the NPV alpha baculovirus clade Ia genome from which above indicated CDS, 5′-UTRs, spacers and/or hr regions are deleted is based on the genome of a baculovirus selected from the group consisting of AcMNPV, PlxyNPV, RoMNPV, BmNPV, MaviMNPV, CfDefMNPV, AgMNPV, EppoNPV, AnpeNPV, CfMNPV, OpMNPV, and HycuNPV.

Exemplary genomes of these baculoviruses are accessible at the NIH and EBI databank. The phrase “based on the genome” means that the native genome of the respectively indicated baculovirus is used as a reference point and that the genome of the invention has that nucleotide sequence sans the CDS and/or 5′UTRs and preferably also spacers of the genes odv-e66, p43, odv-no42 or odv-e56, ptp, bro, ctx, orf603, polyhedrin, egt, bv/odv-e26, ac18, pif-2, env-prot, iap-1, sod, fgf, vubi, gp37, ac69, iap-2, pnk/pn1, ac91, odv-e28 pif-4, pif-3, pif-1, pk-2, chiA, v-cath, pp34, 94K, p26, p10, p74, ac145, and ac150 and/or hr regions.

To determine the extent of the deletion of the genome of the invention a reference genome is used, which is referred to as “native NPV-alpha baculovirus clade Ia genome”. This term is used to designate the genome of a naturally occurring NPV alpha baculovirus clade Ia virus and includes all silent mutations within open reading frames that do not impair functionality of the DNA elements. Preferably, this term comprises NPV-alphabaculovirus clade I a/b genomic sequences that exhibit at least 90% sequence identity to the nucleotide sequence of naturally occurring NPV alpha baculovirus clade Ia genome, e.g. those accessible at the NIH or EBI databank. It is preferred that the nucleotide sequence of the naturally occurring NPV alpha baculovirus clade Ia genome is for: (i) AcMNPV as set out in SEQ ID NO: 1 (NC_{—}001623) with a length of 133,894 base pairs, (ii) PlxyNPV as set out in SEQ ID NO: 2 (NC_{—}008349) with a length of 133,417 base pairs, (iii) RoMNPV as set out in SEQ ID NO: 3 (NC_{—}004323) with a length of 131,526 base pairs, (iv) BmNPV as set our in SEQ ID NO: 4 (NC_{—}001962) with a length of 128,413 base pairs, (v) MaviMNPV as set out in SEQ ID NO: 5 (NC_{—}008725.1) with a length of base pairs 111,953, (vi) CfDefMNPV as set out in SEQ ID NO: 6 (NC_{—}005137.2) with a length of 131,160 base pairs, (vii) AgMNPV as set out in SEQ ID NO: 7 (NC_{—}008520.1) with a length of 132,239 base pairs, (viii) EppoNPV as set out in SEQ ID NO: 8 (NC_{—}003083.1) with a length of 118,584 base pairs, (ix) AnpeNPV as set out in SEQ ID NO: 9 (NC_{—}008035.3) with a length of 126,629 base pairs, (x) CfMNPV as set out in SEQ ID NO: 10 (NC_{—}004778.3) with a length of 129,593 base pairs, (xi) OpMNPV as set out in SEQ ID NO: 11 (NC_{—}001875.2) with a length of 131,995 base pairs, and (xii) HycuNPV as set out in SEQ ID NO: 12 (NC_{—}007767.1) with a length of 132,959 base pairs. Accordingly, the reference native NPV-alpha baculovirus clade Ia genome preferably has at least 90% sequence identity to one of the sequences selected from the group consisting of SEQ ID NO: 1, 2, 3, 4, 5, 6, 7, 8, 9, 10, 11, or 12, more preferably at least 92% sequence identity to one of the sequences selected from the group consisting of SEQ ID NO: 1, 2, 3, 4, 5, 6, 7, 8, 9, 10, 11, or 12, more preferably at least 94% sequence identity to one of the sequences selected from the group consisting of SEQ ID NO: 1, 2, 3, 4, 5, 6, 7, 8, 9, 10, 11, or 12, more preferably at least 96% sequence identity to one of the sequences selected from the group consisting of SEQ ID NO: 1, 2, 3, 4, 5, 6, 7, 8, 9, 10, 11, or 12, even more preferably at least 98% sequence identity to one of the sequences selected from the group consisting of SEQ ID NO: 1, 2, 3, 4, 5, 6, 7, 8, 9, 10, 11, or 12 and most preferably 100% sequence identity to one of the sequences selected from the group consisting of SEQ ID NO: 1, 2, 3, 4, 5, 6, 7, 8, 9, 10, 11, or 12. The most preferred genome is that of AcMNPV according to SEQ ID NO: 1.

The present inventors have found that the CDS indicated in Table 4a and 4b can be deleted without detrimental effect on the respective NPV alpha baculovirus clade Ia virus (all CDS and proteins are with reference to AcMNPV). These CDS are considered to belong to a group that is referred to as Type IT. Accordingly, the deletion of these CDS (and preferably also the respective 5′-UTR, spacer and/or hr regions) are referred to as Type II deletions:

TABLE 4a
AcMNPV

| Name | UniProt | Definition | aa | bp | Start | Stop | Strand |
|---|---|---|---|---|---|---|---|
| ac11 | P41421 | AcOrf-11 peptide | 340 | 1023 | 7899 | 8921 | minus |
| ac30 | P41434 | Similarity to MSV tryptophan | 463 | 1392 | 24315 | 25706 | minus |
| | | repeat family peptide | | | | | |
| Gta | P41447 | global transactivator-like protein | 506 | 1521 | 34010 | 35530 | plus |
| ac63 | P41466 | AcOrf-63 peptide | 155 | 468 | 50795 | 51262 | plus |
| 15k | P41478 | p15 | 126 | 381 | 74356 | 74736 | plus |
| ac97 | P41657 | AcOrf-97 peptide | 56 | 171 | 84839 | 85009 | plus |
| ac121 | P41674 | AcOrf-121 peptide | 58 | 177 | 102647 | 102823 | plus |
| ac140 | P41699 | AcOrf-140 peptide | 60 | 183 | 122625 | 122807 | plus |
| ac146 | P41704 | AcOrf-146 peptide | 201 | 606 | 126527 | 127132 | minus |
| ac149 | P41706 | AcOrf-149 peptide | 107 | 324 | 130167 | 130490 | minus |

TABLE 4b
BmNPV

| Name | UniProt | Definition | aa | bp | Start | Stop | Strand |
|---|---|---|---|---|---|---|---|
| bm4 ac11 | 1488634 | AcMNPV | 340 | 1023 | 3248 | 4270 | minus |
| | | orf11 | | | | | |
| bm51 ac63 | 1488683 | AcMNPV | 155 | 468 | 45935 | 46402 | plus |
| | | orf63 | | | | | |
| 15K | 1488702 | P15 | 126 | 381 | 66329 | 66709 | plus |
| bm98a | 1488731 | AcMNPV | 67 | 174 | 94474 | 94647 | plus |
| ac121 | | orf121 | | | | | |
| bm122 | 1488754 | AcMNPV | 201 | 606 | 116323 | 116928 | minus |
| ac146 | | orf146 | | | | | |
| bm125 | 1488757 | AcMNPV | 106 | 321 | 119993 | 120313 | minus |
| ac149 | | orf149 | | | | | |

The total length of the CDS encoding these proteins is approximately 6,246 base pairs in AcMNPV according to SEQ ID NO: 1. Thus, the native AcMNPV genome may be shortened by deletion of one or more, preferably of all of the CDS encoding the proteins of Table 4a. If all these CDSs are deleted from the AcMNPV genome, the genome of the invention is preferably at least 6,246 base pairs shorter than the native AcMNPV genome. The respective shortening attributable to the deletion of one specific CDS can be derived from the column labelled “bp”. These CDS correspond to approximately 4.7% of the native genome of AcMNPV.

The native BmNPV genome may be shortened by deletion of one or more, preferably of all of the CDS encoding the proteins of Table 4b. If all these CDSs are deleted from the BmNPV genome, the genome of the invention is preferably at least 2,967 base pairs shorter than the native BmNPV genome. The respective shortening attributable to the deletion of one specific CDS can be derived from the column labelled “bp”. These CDS correspond to approximately 2.3% of the native genome of BmNPV.

It is more preferred that one or more of the 5′-UTRs of the CDS are also deleted. In AcMNPV the 5′-UTRs of these CDS have a length of 446 base pairs. In BmNPV the 5′-UTRs of these CDS have a length of 19 base pairs. The following Table 5a and 5b indicate the position of the 5′UTRs of the CDS deleted in preferred genomes of the invention. The 5′-UTR precedes the respectively indicated CDS Start codon by the indicated number of base pairs:

TABLE 5a
AcMNPV

| Name | GeneID | Start | Stop | Strand | Bp |
|---|---|---|---|---|---|
| ac11 | 1403843 | 7899 | 8921 | minus | 36 |
| ac30 | 1403862 | 24315 | 25706 | minus | 113 |
| gta | 1403874 | 34010 | 35530 | plus | 83 |
| ac63 | 1403896 | 50795 | 51262 | plus | 60 |
| 15K | 1403920 | 74356 | 74736 | plus | 140 |
| ac97 | 1403930 | 84839 | 85009 | plus | −29 |
| ac121 | 1403954 | 102647 | 102823 | plus | 11 |
| ac140 | 1403973 | 122625 | 122807 | plus | 70 |
| ac146 | 1403979 | 126527 | 127132 | minus | 17 |
| ac149 | 1403982 | 130167 | 130490 | minus | −35 |

TABLE 5b
BmNPV

| Name | GeneID | Start | Stop | Strand | Bp |
|---|---|---|---|---|---|
| bm4 ac11 | 1488634 | 3248 | 4270 | minus | 25 |
| bm51 ac63 | 1488683 | 45935 | 46402 | plus | 76 |
| 15K | 1488702 | 66329 | 66709 | plus | 4 |
| bm98a ac121 | 1488731 | 94474 | 94647 | plus | −108 |
| bm122 ac146 | 1488754 | 116323 | 116928 | minus | −6 |
| bm125 ac149 | 1488757 | 119993 | 120313 | minus | 28 |

The deletion of one or more of these 5′-UTRs from the genome of a NPV alpha baculovirus clade Ia virus leads to a further reduction of the size of the genome in comparison to the native genome. The deletion of one or more of these 5′-UTRs from the genome of a AcMNPV virus leads to a further reduction of the size of the genome in comparison to the native genome of up to 0.35%. The deletion of one or more of these 5′-UTRs from the genome of a BmNPV virus leads to a further reduction of the size of the genome in comparison to the native genome of up to 0.02%.

Alternatively or additionally the spacers of the AcMNPV CDS may be deleted, which amount to an additional deletion of 2,543 bp. In BmNPV the spacers of these CDS have a length of 3,028 base pairs. With reference to the nucleotide sequence of AcMNPV the spacers that may be deleted are the following:

TABLE 6a
AcMNPV

| Gene-Before | Gene-After | Start | Stop | Bp |
|---|---|---|---|---|
| Ac30 | sod | 25707 | 25819 | 113 |
| hr1a | Ac11 | 7865 | 7898 | 34 |
| Ac41 | GTA | 33927 | 34009 | 83 |
| GTA | Ac43 | 35531 | 35543 | 13 |
| lef9 | Ac63 | 50735 | 50794 | 60 |
| Ac63 | gp37 | 51263 | 51282 | 20 |
| Ac97 | 38K | 85010 | 85020 | 11 |
| hr4c | Ac121 | 102636 | 102646 | 11 |
| ME53 | Ac140 | 122555 | 122624 | 70 |
| Ac140 | IE-01 | 122808 | 122831 | 24 |
| Ac146 | IE-01 exon-2 | 127133 | 127149 | 17 |
| odv-e56 | Ac149 | 130139 | 130166 | 28 |

TABLE 6b
BmNPV

| Gene-After | Gene-before | Start | Stop | bp |
|---|---|---|---|---|
| pk-1 | bm4 ac11 | 3223 | 3247 | 25 |
| lef-9 | bm51 ac63 | 45875 | 45934 | 60 |
| hr3a | p15 | 65574 | 66328 | 755 |
| bm98 ac120 | hr4a | 94372 | 94425 | 54 |
| bm122 ap146 | ie-1 | 116929 | 116993 | 65 |
| odv-e56 | bm125 | 119965 | 119992 | 28 |
| | ac149 | | | |

The deletion of these spacers from the genome of a NPV alpha baculovirus clade Ia virus leads to a further reduction of the size of the genome in comparison to the native genome. Accordingly, in an even more preferred embodiment the size of the genome of the AcMNPV virus of the invention described above is reduced by up to a further 1.89%. In further preferred embodiments, the size of the genome of the BmNPV virus of the invention is reduced up to a further 2.36%

Accordingly, in a preferred embodiment the NPV alpha baculovirus clade Ia genome of the present invention comprises Type I deletions in addition to the type II deletions.

The AcMNPV genome of the invention is preferably at least 30.23% shorter than the native AcMNPV genome (if only Type I and II CDS are deleted), more preferably 35.16% shorter (if Type I and II CDS and 5′-UTRs are deleted) and more preferably 37.05% shorter (if Type I and II CDS, 5′-UTR and spacers are deleted).

The BmNPV genome of the invention is preferably at least 21.62% shorter than the native BmNPV genome (if only Type I and II CDS are deleted), more preferably 22.07% shorter (if Type I and II CDS and 5′-UTRs are deleted) and more preferably 24.38% shorter (if Type I and II CDS, 5′-UTR and spacers are deleted).

The present inventors have found that the CDS indicated in Table 7a and 7b can be deleted without detrimental effect on the respective NPV alpha baculovirus clade Ia virus (all CDS and proteins are with reference to AcMNPV). These CDS are considered to belong to a group that is referred to as Type II. Accordingly, the deletion of these CDS (and preferably also the respective 5′-UTR, spacer and/or hr regions) are referred to as Type III deletions:

TABLE 7a
AcMNPV

| Name | UniProt | Definition | aa | bp | Start | Stop | Strand |
|---|---|---|---|---|---|---|---|
| hispP | P21286 | putative | 182 | 549 | 27733 | 28281 | minus |
| | | histidinol- | | | | | |
| | | phosphalase | | | | | |
| ac44 | P41449 | AcOrf-44 | 131 | 396 | 35758 | 36153 | plus |
| | | peptide | | | | | |
| ac57 | P41461 | AcOrf-57 | 161 | 486 | 47073 | 47558 | plus |
| | | peptide | | | | | |
| ac84 | P41474 | AcOrf-84 | 188 | 567 | 71165 | 71731 | plus |
| | | peptide | | | | | |
| ac112/ | P41665 | AcOrf-112 | 87 | 264 | 96521 | 96784 | plus |
| 113 Nt | | peptide | | | | | |
| ac112/ | P41666 | AcOrf-113 | 169 | 510 | 96789 | 97298 | plus |
| 113 Ct | | peptide | | | | | |
| ac118 | P41671 | AcOrf-118 | 157 | 474 | 100231 | 100704 | minus |
| | | peptide | | | | | |
| ac122 | P41675 | AcOrf-122 | 62 | 189 | 102713 | 102901 | minus |
| | | peptide | | | | | |
| ie-01 | P11138 | putative | 636 | 1911 | 122832 | 128946 | plus |
| | | early gene | | | | | |
| | | transactivator | | | | | |
| ac152 | P41708 | AcOrf-152 | 92 | 279 | 132109 | 132387 | minus |
| | | peptide | | | | | |

TABLE 7b
BmNPV

| Name | UniProt | Definition | aa | bp | Start | Stop | Strand |
|---|---|---|---|---|---|---|---|
| bm35 ac44 | 1488666 | AcMNPV | 131 | 396 | 31903 | 32298 | plus |
| | | orf44 | | | | | |
| odv-e66 | 1488668 | ODV-E66 | 702 | 2109 | 32866 | 34974 | plus |
| bm46 ac57 | 1488678 | AcMNPV | 161 | 486 | 42221 | 42706 | plus |
| | | orf57 | | | | | |
| bm99 ac122 | 1488732 | AcMNPV | 61 | 186 | 94540 | 94725 | minus |
| | | orf122 | | | | | |

The total length of the CDS encoding these proteins is approximately 5,625 base pairs in AcMNPV according to SEQ ID NO: 1. Thus, the native AcMNPV genome may be shortened by deletion of one or more, preferably of all of the CDS encoding these proteins. If all these CDSs are deleted from the AcMNPV genome, the genome of the invention is preferably at least 5,625 base pairs shorter than the native AcMNPV genome. The respective shortening attributable to the deletion of one specific CDS can be derived from the column labelled “bp”. These CDS correspond to approximately 4.2% of the native genome of AcMNPV.

The total length of the CDS encoding these proteins is approximately 3,173 base pairs in BmNPV. Thus, the native BmNPV genome may be shortened by deletion of one or more, preferably of all of the CDS encoding these proteins. If all these CDSs are deleted from the BmNPV genome, the genome of the invention is preferably at least 3,173 base pairs shorter than the native BmNPV genome. The respective shortening attributable to the deletion of one specific CDS can be derived from the column labelled “bp”. These CDS correspond to approximately 2,47% of the native genome of BmNPV.

Accordingly, in a preferred embodiment the NPV alpha baculovirus clade Ia genome of the present invention comprises (i) Type II deletions, (ii) Type I deletions and Type III deletions, (iii) Type II and Type III deletions or (iv) Type I, II and III deletions.

The AcMNPV genome of the invention is preferably at least 25.66% shorter than the native AcMNPV genome (if only Type I CDS are deleted and Type II CDS, 5′-UTRs and spacers are retained), more preferably 27.24% shorter (if Type I CDS and 5′-UTRs are deleted and Type II CDS, 5′-UTRs and spacers are retained) and more preferably 34.14% shorter (if Type I and II CDS, 5′-UTR and spacers are deleted).

The BmNPV genome of the invention is preferably at least 18.3% shorter than the native BmNPV genome (if only Type I CDS are deleted and Type II CDS, 5′-UTRs and spacers are retained), more preferably 19.55% shorter (if Type I CDS and 5′-UTRs are deleted and Type IT CDS, 5′-UTRs and spacers are retained) and more preferably 24.20% shorter (if Type I and II CDS, 5′-UTR and spacers are deleted).

It is more preferred that the 5′-UTRs of these CDS are also deleted. In AcMNPV the 5′-UTRs of these CDS have a length of 351 base pairs. In BmNPV the 5′-UTRs of these CDS have a length of 7 base pairs. The following Tables 8a and 8b indicate the position of the 5′UTRs of the CDS deleted in preferred genomes of the invention. The 5′-UTR precedes the respectively indicated CDS Start codon by the indicated number of base pairs:

TABLE 8a
AcMNPV

| Name | GeneID | Start | Stop | Strand | Bp |
|---|---|---|---|---|---|
| hisP | 1403865 | 27733 | 28281 | minus | 12 |
| ac44 | 1403876 | 35758 | 36153 | plus | −20 |
| ac57 | 1403890 | 47073 | 47558 | plus | 184 |
| ac84 | 1403917 | 71165 | 71731 | plus | 31 |
| ac112/113 Nt | 1403945 | 96521 | 96784 | plus | 169 |
| ac112/113 Ct | 1403946 | 96789 | 97298 | plus | 4 |
| ac118 | 1403951 | 100231 | 100704 | minus | −6 |
| ac122 | 1403955 | 102713 | 102901 | minus | 62 |
| ie-01 | 1403988 | 122832 | 128946 | plus | 24 |
| ac152 | 1403985 | 132109 | 132387 | minus | 138 |

TABLE 8b
BmNPV

| Name | GeneID | Start | Stop | Strand | Bp |
|---|---|---|---|---|---|
| bm35 ac44 | 1488666 | 31903 | 32298 | plus | 1 |
| odv-e66 | 1488668 | 32866 | 34974 | plus | 98 |
| bm46 ac57 | 1488678 | 42221 | 42706 | plus | 16 |
| bm99 ac122 | 1488732 | 94540 | 94725 | minus | −108 |

The deletion of one or more of these 5′-UTRs from the genome of a AcMNPV leads to a further reduction of the size of the genome in comparison to the native genome of up to 0.26%.

The deletion of one or more of these 5′-UTRs from the genome of a BmNPV leads to a further reduction of the size of the genome in comparison to the native genome of up to 0.01%. Alternatively or additionally the spacers of the CDS in AcMNPV may be deleted, which amount to an additional deletion of 2183 bp. In BmNPV the spacers of these CDS have a length of 3021 base pairs. With reference to the nucleotide sequence of AcMNPV the spacers that may be deleted are the following:

TABLE 9a
AcMNPV

| Gene-After | Gene-Before | Start | Stop | Bp |
|---|---|---|---|---|
| fgt | HisP | 27587 | 27732 | 146 |
| HisP | Ac34 | 28282 | 28293 | 12 |
| Ac44 | Ac45 | 36154 | 36154 | 1 |
| Ac57 | Ac58 | 47559 | 47573 | 15 |
| hr3 | Ac84 | 71134 | 71164 | 31 |
| Ac112 | Ac113 | 96785 | 96788 | 4 |
| Ac117 | Ac118 | 100198 | 100230 | 33 |
| Ac122 | pk-2 | 102902 | 102963 | 62 |
| Ac140 | IE-01 | 122808 | 122831 | 24 |
| IE-2 | Ac152 | 132084 | 132108 | 25 |

TABLE 9b
BmNPV

| Gene-After | Gene-before | Start | Stop | bp |
|---|---|---|---|---|
| bm35 ac44 | bm36 ac45 | 32299 | 32299 | 1 |
| odv-e66 | ets | 34975 | 35072 | 98 |
| bm45 ac56 | bm46 ac57 | 41969 | 42220 | 252 |
| bm46 ac57 | bm47 ac58 | 42707 | 42722 | 16 |
| bm99 ac122 | gcn2 | 94726 | 94758 | 33 |

The deletion of these spacers from the genome of a AcMNPV virus leads to a further reduction of the size of the genome in comparison to the native genome of 1.63%. Accordingly, in an even more preferred embodiment the size of the genome of the AcMNPVvirus of the invention is reduced by up to a further 1.63%.

The deletion of these spacers from the genome of a BmNPV virus leads to a further reduction of the size of the genome in comparison to the native genome of 2.35%. Accordingly, in an even more preferred embodiment the size of the genome of the BmNPVvirus of the invention is reduced by up to a further 2.35%.

The present inventors have found that the CDS indicated in Table 10a and 10b can be deleted without detrimental effect on the respective NPV alpha baculovirus clade Ia virus (all CDS and proteins are with reference to AcMNPV). These CDS are considered to belong to a group that is referred to as Type IV. Accordingly, the deletion of these CDS (and preferably also the respective 5′-UTR, spacer and/or hr regions) are referred to as Type IV deletions:

TABLE 10a
AcMNPV

| Name | UniProt | Definition | aa | bp | Start | Stop | Strand |
|---|---|---|---|---|---|---|---|
| Pcna | P11038 | proliferating cell | 256 | 771 | 39643 | 40413 | minus |
| | | nuclear antigen | | | | | |
| ac56 | P41462 | AcOrf-56 peptide | 84 | 255 | 46634 | 46888 | plus |
| hcf-1 | P41470 | AcOrf-70 peptide; | 290 | 873 | 60110 | 60982 | plus |
| | | host cell factor-1 | | | | | |
| ac85 | P41475 | AcOrf-85 peptide | 53 | 162 | 71934 | 72095 | plus |
| cg30 | P16091 | hypothetical protein | 264 | 795 | 74737 | 75531 | minus |
| | | ACNVgp089 | | | | | |
| ac116 | P41669 | AcOrf-116 peptide | 56 | 171 | 99804 | 99974 | minus |
| ac117 | P41670 | AcOrf-117 peptide | 95 | 288 | 99910 | 100197 | Plus |

TABLE 10b
BmNPV

| Name | UniProt | Definition | aa | bp | Start | Stop | Strand |
|---|---|---|---|---|---|---|---|
| bm45 | 1488677 | AcMNPV orf56 | 84 | 255 | 41714 | 41968 | plus |
| ac56 | | | | | | | |
| cg30 | 1488703 | CG30 | 267 | 804 | 66714 | 67517 | minus |
| bm95a | 1488727 | AcMNPV orf116 | 56 | 171 | 92007 | 92177 | minus |
| ac116 | | | | | | | |
| bm96 | 1488728 | AcMNPV orf117 | 95 | 288 | 92113 | 92400 | plus |
| ac117 | | | | | | | |

The total length of the CDS encoding these proteins is approximately 3,027 base pairs in AcMNPV and approximately 1,230 base pairs in BmNPV. Thus, the native NPV alpha baculovirus clade Ia genome may be shortened by deletion of one or more, preferably of all of the CDS encoding these proteins.

If all these CDSs are deleted from the AcMNPV genome, the genome of the invention is preferably at least 3,027 base pairs shorter than the native AcMNPV genome. The respective shortening attributable to the deletion of one specific CDS can be derived from the column labelled “bp”. These CDS correspond to approximately 2.26% of the native genome of AcMNPV.

If all these CDSs are deleted from the BmNPV genome, the genome of the invention is preferably at least 823 base pairs shorter than the native BmNPV genome. The respective shortening attributable to the deletion of one specific CDS can be derived from the column labelled “bp”. These CDS correspond to approximately 0.64% of the native genome of BmMNPV.

It is more preferred that one or more of the 5′-UTRs of these CDS are also deleted. In AcMNPV the 5′-UTRs of these CDS have a length of 446 base pairs. In BmNPV the 5′-UTRs of these CDS have a length of 263 base pairs. The following Table 11a and 11b indicates the position of the 5′UTRs of the CDS deleted in the preferred AcMNPV and BmNPV genomes of the invention, respectively. The 5′-UTR precedes the respectively indicated CDS Start codon by the indicated number of base pairs:

TABLE 11a
AcMNPV

| Name | GeneID | Start | Stop | Strand | Bp |
|---|---|---|---|---|---|
| pcna | 1403881 | 39643 | 40413 | minus | 109 |
| ac56 | 1403889 | 46634 | 46888 | plus | 1 |
| hcf-1 | 1403903 | 60110 | 60982 | plus | 45 |
| ac85 | 1403918 | 71934 | 72095 | plus | 202 |
| cg30 | 1403921 | 74737 | 75531 | minus | 2 |
| ac116 | 1403949 | 99804 | 99974 | minus | −65 |

TABLE 11b
BmNPV

| Name | GeneID | Start | Stop | Strand | Bp |
|---|---|---|---|---|---|
| bm45 ac56 | 1488677 | 84 | 255 | 41714 | 252 |
| cg30 | 1488703 | 267 | 804 | 66714 | 4 |
| bm95a ac116 | 1488727 | 56 | 171 | 92007 | 7 |

The deletion of these 5′ UTRs from the genome of a AcMNPV virus leads to a further reduction of the size of the genome in comparison to the native genome of up to 0.25%.

The deletion of these 5′-UTRs from the genome of a BmNPV virus leads to a further reduction of the size of the genome in comparison to the native genome of up to 0.20%.

Alternatively or additionally the spacers of the CDS in AcMNPV may be deleted, which amount to an additional deletion of 1,847 bp. In BmNPV the spacers of these CDS have a length of 2,760 base pairs. With reference to the nucleotide sequence of AcMNPV the spacers that may be deleted are the following:

TABLE 12a
AcMNPV

| Gene-After | Gene-Before | Start | Stop | bp |
|---|---|---|---|---|
| Ac48 | Pcna | 39620 | 39642 | 23 |
| pcna | lef8 | 40414 | 40522 | 109 |
| Ac55 | Ac56 | 46633 | 46633 | 1 |
| Ac84 | Ac85 | 71732 | 71933 | 202 |
| Ac115 | Ac116 | 99797 | 99803 | 7 |

TABLE 12b
BmNPV

| Gene-After | Gene-before | Start | Stop | bp |
|---|---|---|---|---|
| bm45 ac56 | bm46 ac57 | 41969 | 42220 | 252 |
| p30 | vp39 | 67518 | 67519 | 2 |
| bm95 ac115 | bm95a ac116 | 92000 | 92006 | 7 |

The deletion of these spacers from the genome of a NPV alpha baculovirus clade Ia virus leads to a further reduction of the size of the genome in comparison to the native genome. Accordingly, in an even more preferred embodiment the size of the genome of the AcMNPV virus of the invention is reduced by up by a further 1.38%. In further preferred embodiments the size of the genome of the BmNPV virus is reduced 2.15%

In a preferred embodiment the NPV alpha baculovirus clade Ia genome, preferably the AcMNPV or the BmNPV genome, of the invention further lacks all 5′-UTR and/or 3′-UTR of the genes of (i) Type I, (ii) Type I and Type II, (iii) Type I and Type III, (iv) Type I and Type IV, (v) Type II and Type III, (vi) Type II and Type IV, (vii) Type II and Type IV, (viii) Type I, Type II and Type I, (ix) Type I, Type II and Type IV, (x) Type I, Type III and Type IV, (xi) Type II, Type I and Type IV, or (xii) Type I, Type II, Type III and Type IV.

In a preferred embodiment the NPV alpha baculovirus clade Ia genome, preferably the AcMNPV or the BmNPV genome, of the invention further lacks the spacers 5′ and/or 3′ of the genes of (i) Type I, (ii) Type I and Type II, (iii) Type I and Type m, (iv) Type I and Type IV, (v) Type II and Type I, (vi) Type II and Type IV, (vii) Type III and Type IV, (viii) Type I, Type II and Type II, (ix) Type I, Type II and Type IV, (x) Type I, Type III and Type IV, (xi) Type II, Type I and Type IV, or (xii) Type I, Type II, Type III and Type IV.

In a preferred embodiment the NPV alpha baculovirus clade Ia genome, preferably the AcMNPV or the BmNPV genome, of the invention further lacks one or more of the heterologous repeat (HR) sequences.

The preferred NPV alpha baculovirus clade Ia genomes, preferrably the AcMNPV or the BmNPV genome, lack the following heterologous repeat sequences:

(i) for AcMNPV one or more, preferably all of the heterologous repeat sequences corresponding to the regions spanning nucleotides 1 to 445, 7,747 to 7,864, 26,293 to 26,961, 48,679 to 48,708, 70,468 to 71,133, 93,456 to 93,605, 97,396 to 97,881, 102,606 to 102,635, 117,479 to 117,987 and 133,883 to 133,894 of the genome sequence according to SEQ ID NO: 1,

(ii) for PlxyNPV one or more, preferably all of the heterologous repeat sequences corresponding to the regions spanning nucleotides 1 to 441, 7,725 to 7,842, 27,391 to 28,255, 49,952 to 49,981, 50,439 to 51,255, 72,580 to 73,046, 94,061 to 94,210, 98,016 to 98,409, 103,134 to 103,163, 117,943 to 118,456, and 134,406 to 133,417 of the genome sequence according to SEQ ID NO: 2,

(iii) for RoMNPV one or more, preferably all of the heterologous repeat sequences corresponding to the regions spanning nucleotides 1 to 326, 6,454 to 6,491, 24,805 to 25,141, 46,826 to 46,855, 68,628 to 69,279, 91,594 to 91,623, 95,394 to 95,750, 100,470 to 100,499, 115,321 to 115,714, and 131,515 to 131,526 of the genome sequence according to SEQ ID NO: 3,

(iv) for BmNPV one or more, preferably all of the heterologous repeat sequences corresponding to the regions spanning nucleotides, 22,497 to 23,200, 24,012 to 24,278, 29,485 to 29566, 43821 to 43857, 64802 to 65350, 65499 to 65573, 86431 to 86648, 89552 to 90142, 94426 to 94483, 106947 to 107561 and 123706 to 124297 of the genome sequence according to SEQ ID NO: 4,

(v) for MaviMNPV one or more, preferably all of the heterologous repeat sequences corresponding to the regions spanning nucleotides 20436 to 21162, 56264 to 57313, 77148 to 77878, 92877 to 93652, and 109272 to 110071 of the genome sequence according to SEQ ID NO: 5,

(vi) for CfDefMNPV one or more, preferably all of the heterologous repeat sequences corresponding to the regions spanning nucleotides 6450-6723, 15714-16013, 22164 to 22959, 37020 to 37175, 65930 to 65959, 76886 to 77072, 86479 to 86720, 96698 to 96936, 100566 to 100716 and 105456 to 105485 of the genome sequence according to SEQ ID NO: 6, (vii) for AgMNPV one or more, preferably all of the heterologous repeat sequences corresponding to the regions spanning nucleotides 19648 to 19685, 52009 to 52064, 126402 to 126447, 126568 to 127008 and 128754 to 128960 of the genome sequence according to SEQ ID NO: 7,

(viii) for EppoNPV one or more, preferably all of the heterologous repeat sequences corresponding to the regions spanning nucleotides 3992 to 4094, 18956 to 19121, 88438 to 88950, 95415 to 95715, and 103275 to 103722 of the genome sequence according to SEQ ID NO: 8,

(ix) for AnpeNPV one or more, preferably all of the heterologous repeat sequences corresponding to the regions spanning nucleotides 18636 to 19241, 41040 to 41134, 48378 to 48521, 65768 to 65862, 65894 to 66019, 75128 to 75220, 78778 to 79048, 92649 to 92655, 110552 to 110905, 117548 to 117631, 120778 to 120838, 122220 to 122259 and 125492 to 125506 of the genome sequence according to SEQ ID NO: 9,

(x) for CfMNPV one or more, preferably all of the heterologous repeat sequences corresponding to the regions spanning nucleotides 6460 to 6723, 15714 to 16013, 22164 to 22959, 37020 to 37175, 65930 to 65959, 76886 to 77072, 86479 to 86720, 96698 to 96936, 100566 to 100716, 105456 to 105485, 113424 to 113779, 125448 to 125477 and 126985 to 127146 of the genome sequence according to SEQ ID NO: 10,

(xi) for OpMNPV one or more, preferably all of the heterologous repeat sequences corresponding to the regions spanning nucleotides 103528 to 103833, 127459 to 130270 and 141587 to 142185 of the genome sequence according to SEQ ID NO: 11, and

(xii) for HycuNPV one or more, preferably all of the heterologous repeat sequences corresponding to the regions spanning nucleotides 4710 to 5602, 18642 to 19810, 27465 to 28918, 35645 to 36379, 66095 to 66778, and 112869 to 113601 of the genome sequence according to SEQ ID NO: 12.

The relative reductions in genome size of the genomes of the invention that may be achieved in comparison to the native NPV-alpha baculovirus clade Ia genome are indicated in percent in Table 13a and 13b, i.e. the following table indicates preferred reduction in size that are achieved for the genomes of the present invention. The absolute reductions can be calculated for each of the NPV-alpha baculovirus clade Ia genome of the invention on the basis of the lengths of the respective elements indicated exemplary above for AcMNPV.

TABLE 13a
AcMNPV

| | Type I | Type I | Type II | Type II | Type III | Type III | Type IV | Type IV | | Total |
|---|---|---|---|---|---|---|---|---|---|---|
| No. | CDS | 5′-UTR | CDS | 5′-UTR | CDS | 5′-UTR | CDS | 5′-UTR | spacer | Reduction |
| 1 | 25.66 | | | | | | | | | 25.66 |
| 2 | 25.66 | 1.58 | | | | | | | | 27.24 |
| 3 | 25.66 | 1.58 | | | | | | | 2.24 | 29.48 |
| 4 | | | 4.66 | | | | | | | 4.66 |
| 5 | | | 4.66 | 0.35 | | | | | | 5.01 |
| 6 | | | 4.66 | 0.35 | | | | | 1.89 | 6.90 |
| 7 | | | | | 4.2 | | | | | 4.20 |
| 8 | | | | | 4.2 | 0.26 | | | | 4.46 |
| 9 | | | | | 4.2 | 0.26 | | | 1.98 | 6.44 |
| 10 | | | | | | | 2.26 | | | 2.26 |
| 11 | | | | | | | 2.26 | 0.25 | | 2.51 |
| 12 | | | | | | | 2.26 | 0.25 | 1.99 | 4.50 |
| 13 | 25.66 | | 4.66 | | | | | | | 30.32 |
| 14 | 25.66 | | 4.66 | 0.35 | | | | | | 30.67 |
| 15 | 25.66 | | 4.66 | 0.35 | | | | | 1.89 | 32.56 |
| 16 | 25.66 | | | | 4.2 | | | | | 29.86 |
| 17 | 25.66 | | | | 4.2 | 0.26 | | | | 30.12 |
| 18 | 25.66 | | | | 4.2 | 0.26 | | | 1.98 | 32.10 |
| 19 | 25.66 | | | | | | 2.26 | | | 27.92 |
| 20 | 25.66 | | | | | | 2.26 | 0.25 | | 28.17 |
| 21 | 25.66 | | | | | | 2.26 | 0.25 | 1.99 | 30.16 |
| 22 | 25.66 | 1.58 | 4.66 | | | | | | | 31.90 |
| 23 | 25.66 | 1.58 | 4.66 | 0.35 | | | | | | 32.25 |
| 24 | 25.66 | 1.58 | 4.66 | 0.35 | | | | | 1.89 | 34.14 |
| 25 | 25.66 | 1.58 | | | 4.2 | | | | | 31.44 |
| 26 | 25.66 | 1.58 | | | 4.2 | 0.26 | | | | 31.70 |
| 27 | 25.66 | 1.58 | | | 4.2 | 0.26 | | | 1.98 | 33.68 |
| 28 | 25.66 | 1.58 | | | | | 2.26 | | | 29.50 |
| 29 | 25.66 | 1.58 | | | | | 2.26 | 0.25 | | 29.75 |
| 30 | 25.66 | 1.58 | | | | | 2.26 | 0.25 | 1.99 | 31.74 |
| 31 | 25.66 | 1.58 | 4.66 | | | | | | | 31.90 |
| 32 | 25.66 | 1.58 | 4.66 | 0.35 | | | | | | 32.25 |
| 33 | 25.66 | 1.58 | 4.66 | 0.35 | | | | | 1.89 | 34.14 |
| 34 | 25.66 | 1.58 | | | 4.2 | | | | | 31.44 |
| 35 | 25.66 | 1.58 | | | 4.2 | 0.26 | | | | 31.70 |
| 36 | 25.66 | 1.58 | | | 4.2 | 0.26 | | | 1.98 | 33.68 |
| 37 | 25.66 | 1.58 | | | | | 2.26 | | | 29.50 |
| 38 | 25.66 | 1.58 | | | | | 2.26 | 0.25 | | 29.75 |
| 39 | 25.66 | 1.58 | | | | | 2.26 | 0.25 | 1.99 | 31.74 |
| 40 | 25.66 | 1.58 | 4.66 | | 4.2 | | | | | 36.10 |
| 41 | 25.66 | 1.58 | 4.66 | | 4.2 | 0.26 | | | | 36.36 |
| 42 | 25.66 | 1.58 | 4.66 | | 4.2 | 0.26 | | | 1.98 | 38.34 |
| 43 | 25.66 | 1.58 | 4.66 | | | | 2.26 | | | 34.16 |
| 44 | 25.66 | 1.58 | 4.66 | | | | 2.26 | 0.25 | | 34.41 |
| 45 | 25.66 | 1.58 | 4.66 | | | | 2.26 | 0.25 | 1.99 | 36.40 |
| 46 | 25.66 | 1.58 | 4.66 | 0.35 | 4.2 | | | | | 36.45 |
| 47 | 25.66 | 1.58 | 4.66 | 0.35 | 4.2 | 0.26 | | | | 36.71 |
| 48 | 25.66 | 1.58 | 4.66 | 0.35 | 4.2 | 0.26 | | | 1.63 | 38.34 |
| 49 | 25.66 | 1.58 | 4.66 | 0.35 | | | 2.26 | | | 34.51 |
| 50 | 25.66 | 1.58 | 4.66 | 0.35 | | | 2.26 | 0.25 | | 34.76 |
| 51 | 25.66 | 1.58 | 4.66 | 0.35 | | | 2.26 | 0.25 | 1.64 | 36.40 |
| 52 | 25.66 | 1.58 | 4.66 | 0.35 | 4.2 | | | | | 36.45 |
| 53 | 25.66 | 1.58 | 4.66 | 0.35 | 4.2 | 0.26 | | | | 36.71 |
| 54 | 25.66 | 1.58 | 4.66 | 0.35 | 4.2 | 0.26 | | | 1.63 | 38.34 |
| 55 | 25.66 | 1.58 | 4.66 | 0.35 | | | 2.26 | | | 34.51 |
| 56 | 25.66 | 1.58 | 4.66 | 0.35 | | | 2.26 | 0.25 | | 34.76 |
| 57 | 25.66 | 1.58 | 4.66 | 0.35 | | | 2.26 | 0.25 | 1.64 | 36.40 |
| 58 | 25.66 | 1.58 | 4.66 | 0.35 | 4.2 | | 2.26 | | | 38.71 |
| 59 | 25.66 | 1.58 | 4.66 | 0.35 | 4.2 | | 2.26 | 0.25 | | 38.96 |
| 60 | 25.66 | 1.58 | 4.66 | 0.35 | 4.2 | | 2.26 | 0.25 | 1.64 | 40.60 |
| 61 | 25.66 | 1.58 | 4.66 | 0.35 | 4.2 | 0.26 | 2.26 | | | 38.97 |
| 62 | 25.66 | 1.58 | 4.66 | 0.35 | 4.2 | 0.26 | 2.26 | 0.25 | | 39.22 |
| 63 | 25.66 | 1.58 | 4.66 | 0.35 | 4.2 | 0.26 | 2.26 | 0.25 | 1.38 | 40.60 |
| 64 | 25.66 | 1.58 | 4.66 | 0.35 | 4.2 | 0.26 | 2.26 | | | 38.97 |
| 65 | 25.66 | 1.58 | 4.66 | 0.35 | 4.2 | 0.26 | 2.26 | 0.25 | | 39.22 |
| 66 | 25.66 | 1.58 | 4.66 | 0.35 | 4.2 | 0.26 | 2.26 | 0.25 | 1.38 | 40.60 |
| 67 | 25.66 | 1.58 | | | 4.2 | | 2.26 | | | 33.70 |
| 68 | 25.66 | 1.58 | | | 4.2 | | 2.26 | 0.25 | | 33.95 |
| 68 | 25.66 | 1.58 | | | 4.2 | | 2.26 | 0.25 | 1.99 | 35.94 |
| 69 | 25.66 | 1.58 | | | 4.2 | 0.26 | 2.26 | | | 33.96 |
| 70 | 25.66 | 1.58 | | | 4.2 | 0.26 | 2.26 | 0.25 | | 34.21 |
| 71 | 25.66 | 1.58 | | | 4.2 | 0.26 | 2.26 | 0.25 | 1.73 | 35.94 |
| 72 | 25.66 | 1.58 | | | 4.2 | 0.26 | 2.26 | | | 33.96 |
| 73 | 25.66 | 1.58 | | | 4.2 | 0.26 | 2.26 | 0.25 | | 34.21 |
| 74 | 25.66 | 1.58 | | | 4.2 | 0.26 | 2.26 | 0.25 | 1.73 | 35.94 |

TABLE 13b
BmNPV

| | Type I | Type I | Type II | Type II | Type III | Type III | Type IV | Type IV | | Total |
|---|---|---|---|---|---|---|---|---|---|---|
| No. | CDS | 5′-UTR | CDS | 5′-UTR | CDS | 5′-UTR | CDS | 5′-UTR | spacer | Reduction |
| 1 | 18.3 | | | | | | | | | 18.30 |
| 2 | 18.3 | 1.25 | | | | | | | | 19.55 |
| 3 | 18.3 | 1.25 | | | | | | | 2.34 | 21.89 |
| 4 | | | 2.31 | | | | | | | 2.31 |
| 5 | | | 2.31 | 0.02 | | | | | | 2.33 |
| 6 | | | 2.31 | 0.02 | | | | | 2.32 | 4.65 |
| 7 | | | | | 2.47 | | | | | 2.47 |
| 8 | | | | | 2.47 | 0.01 | | | | 2.48 |
| 9 | | | | | 2.47 | 0.01 | | | | 2.48 |
| 10 | | | | | | | 0.64 | | | 0.64 |
| 11 | | | | | | | 0.64 | 0.2 | | 0.84 |
| 12 | | | | | | | 0.64 | 0.2 | 2.14 | 2.98 |
| 13 | 18.3 | | 2.31 | | | | | | | 20.61 |
| 14 | 18.3 | | 2.31 | 0.02 | | | | | | 20.63 |
| 15 | 18.3 | | 2.31 | 0.02 | | | | | 2.32 | 22.95 |
| 16 | 18.3 | | | | 2.47 | | | | | 20.77 |
| 17 | 18.3 | | | | 2.47 | 0.01 | | | | 20.78 |
| 18 | 18.3 | | | | 2.47 | 0.01 | | | 2.33 | 23.11 |
| 19 | 18.3 | | | | | | 0.64 | | | 18.94 |
| 20 | 18.3 | | | | | | 0.64 | 0.2 | | 19.14 |
| 21 | 18.3 | | | | | | 0.64 | 0.2 | 2.14 | 21.28 |
| 22 | 18.3 | 1.25 | 2.31 | | | | | | | 21.86 |
| 23 | 18.3 | 1.25 | 2.31 | 0.02 | | | | | | 21.88 |
| 24 | 18.3 | 1.25 | 2.31 | 0.02 | | | | | 2.32 | 24.20 |
| 25 | 18.3 | 1.25 | | | 2.47 | | | | | 22.02 |
| 26 | 18.3 | 1.25 | | | 2.47 | 0.01 | | | | 22.03 |
| 27 | 18.3 | 1.25 | | | 2.47 | 0.01 | | | 2.33 | 24.36 |
| 28 | 18.3 | 1.25 | | | | | 0.64 | | | 20.19 |
| 29 | 18.3 | 1.25 | | | | | 0.64 | 0.2 | | 20.39 |
| 30 | 18.3 | 1.25 | | | | | 0.64 | 0.2 | 2.14 | 22.53 |
| 31 | 18.3 | 1.25 | 2.31 | | | | | | | 21.86 |
| 32 | 18.3 | 1.25 | 2.31 | 0.02 | | | | | | 21.88 |
| 33 | 18.3 | 1.25 | 2.31 | 0.02 | | | | | 2.32 | 24.20 |
| 34 | 18.3 | 1.25 | | | 2.47 | | | | | 22.02 |
| 35 | 18.3 | 1.25 | | | 2.47 | 0.01 | | | | 22.03 |
| 36 | 18.3 | 1.25 | | | 2.47 | 0.01 | | | 2.33 | 24.36 |
| 37 | 18.3 | 1.25 | | | | | 0.64 | | | 20.19 |
| 38 | 18.3 | 1.25 | | | | | 0.64 | 0.2 | | 20.39 |
| 39 | 18.3 | 1.25 | | | | | 0.64 | 0.2 | 2.14 | 22.53 |
| 40 | 18.3 | 1.25 | 2.31 | | 2.47 | | | | | 24.33 |
| 41 | 18.3 | 1.25 | 2.31 | | 2.47 | 0.01 | | | | 24.34 |
| 42 | 18.3 | 1.25 | 2.31 | | 2.47 | 0.01 | | | 2.33 | 26.67 |
| 43 | 18.3 | 1.25 | 2.31 | | | | 0.64 | | | 22.50 |
| 44 | 18.3 | 1.25 | 2.31 | | | | 0.64 | 0.2 | | 22.70 |
| 45 | 18.3 | 1.25 | 2.31 | | | | 0.64 | 0.2 | 2.14 | 24.84 |
| 46 | 18.3 | 1.25 | 2.31 | 0.02 | 2.47 | | | | | 24.35 |
| 47 | 18.3 | 1.25 | 2.31 | 0.02 | 2.47 | 0.01 | | | | 24.36 |
| 48 | 18.3 | 1.25 | 2.31 | 0.02 | 2.47 | 0.01 | | | 2.31 | 26.67 |
| 49 | 18.3 | 1.25 | 2.31 | 0.02 | | | 0.64 | | | 22.52 |
| 50 | 18.3 | 1.25 | 2.31 | 0.02 | | | 0.64 | 0.2 | | 22.72 |
| 51 | 18.3 | 1.25 | 2.31 | 0.02 | | | 0.64 | 0.2 | 2.12 | 24.84 |
| 52 | 18.3 | 1.25 | 2.31 | 0.02 | 2.47 | | | | | 24.35 |
| 53 | 18.3 | 1.25 | 2.31 | 0.02 | 2.47 | 0.01 | | | | 24.36 |
| 54 | 18.3 | 1.25 | 2.31 | 0.02 | 2.47 | 0.01 | | | 2.31 | 26.67 |
| 55 | 18.3 | 1.25 | 2.31 | 0.02 | | | 0.64 | | | 22.52 |
| 56 | 18.3 | 1.25 | 2.31 | 0.02 | | | 0.64 | 0.2 | | 22.72 |
| 57 | 18.3 | 1.25 | 2.31 | 0.02 | | | 0.64 | 0.2 | 2.12 | 24.84 |
| 58 | 18.3 | 1.25 | 2.31 | 0.02 | 2.47 | | 0.64 | | | 24.99 |
| 59 | 18.3 | 1.25 | 2.31 | 0.02 | 2.47 | | 0.64 | 0.2 | | 25.19 |
| 60 | 18.3 | 1.25 | 2.31 | 0.02 | 2.47 | | 0.64 | 0.2 | 2.12 | 27.31 |
| 61 | 18.3 | 1.25 | 2.31 | 0.02 | 2.47 | 0.01 | 0.64 | | | 25.00 |
| 62 | 18.3 | 1.25 | 2.31 | 0.02 | 2.47 | 0.01 | 0.64 | 0.2 | | 25.20 |
| 63 | 18.3 | 1.25 | 2.31 | 0.02 | 2.47 | 0.01 | 0.64 | 0.2 | 2.11 | 27.31 |
| 64 | 18.3 | 1.25 | 2.31 | 0.02 | 2.47 | 0.01 | 0.64 | | | 25.00 |
| 65 | 18.3 | 1.25 | 2.31 | 0.02 | 2.47 | 0.01 | 0.64 | 0.2 | | 25.20 |
| 66 | 18.3 | 1.25 | 2.31 | 0.02 | 2.47 | 0.01 | 0.64 | 0.2 | 2.11 | 27.31 |
| 67 | 18.3 | 1.25 | | | 2.47 | | 0.64 | | | 22.66 |
| 68 | 18.3 | 1.25 | | | 2.47 | | 0.64 | 0.2 | | 22.86 |
| 68 | 18.3 | 1.25 | | | 2.47 | | 0.64 | 0.2 | 2.14 | 25.00 |
| 69 | 18.3 | 1.25 | | | 2.47 | 0.01 | 0.64 | | | 22.67 |
| 70 | 18.3 | 1.25 | | | 2.47 | 0.01 | 0.64 | 0.2 | | 22.87 |
| 71 | 18.3 | 1.25 | | | 2.47 | 0.01 | 0.64 | 0.2 | 2.13 | 25.00 |
| 72 | 18.3 | 1.25 | | | 2.47 | 0.01 | 0.64 | | | 22.67 |
| 73 | 18.3 | 1.25 | | | 2.47 | 0.01 | 0.64 | 0.2 | | 22.87 |
| 74 | 18.3 | 1.25 | | | 2.47 | 0.01 | 0.64 | 0.2 | 2.13 | 25.00 |

In each of the cases indicated above in Tables 13a and 13b one or more, preferably all of the hr regions are also deleted. For AcMNPV this leads to a further reduction in size of up to 3,115 base pairs (including overlaps with a CDS at positions 48,679 to 48,708) or up to 3,085 (excluding the overlap with the CDS), which equates to a size reduction of up to 2.33% and 2.30%, respectively. For BmNPV the size reduction of deleting one or more, preferably all of the hr regions leads to a deletion of up to 3,766 base pairs, which equates to a relative size reduction of up to 2.93%.

The present inventors have also discovered that certain genes of the NPV alpha baculovirus clade Ia genome are important to maintain vital functions of the virus. These genes are involved in various aspects, e.g. transcription, replication, assembly, packaging, and infectivity of the virus. It is preferred that these genes are left intact in the genomes of the invention.

Accordingly, in a preferred embodiment the AcMNPV genome of the invention comprises at least one of the genes encoding helicase, 38K, lef-5, 49K and odv-e18+28. Preferably, the genome of the invention comprises all of these genes. These genes are indicated in Table 14a with reference to the genome of AcMNPV and are designated as vital genes of category I:

TABLE 14a
AcMNPV

| Name | UniProt | Definition | aa | bp | Start | Stop | Strand |
|---|---|---|---|---|---|---|---|
| Helicase | P24307 | helicase | 1221 | 3666 | 80694 | 84359 | minus |
| 38K | P24745 | hypothetical protein | 320 | 963 | 85021 | 85983 | minus |
| | | ACNVgp099 | | | | | |
| lef-5 | P41658 | late expression factor 5 | 265 | 798 | 85918 | 86715 | Plus |
| 49K | P41700 | early 49 Daa protein | 477 | 1434 | 123632 | 125065 | Plus |
| odv-e18 + 28 | P41701 | occlusion-derived virus | 62 | 189 | 125153 | 125341 | Plus |
| | | envelope protein | | | | | |

Accordingly, in a preferred embodiment the BmNPV genome of the invention comprises at least one of the genes encoding helicase, 38K, lef-5, 49K and odv-e18+28. Preferably, the genome of the invention comprises all of these genes. These genes are indicated in Table 14b with reference to the genome of BmNPV and are designated as vital genes of category I:

TABLE 14b
BmNPV

| Name | UniProt | Definition | aa | bp | Start | Stop | Strand |
|---|---|---|---|---|---|---|---|
| helicase | 1724488 | DNA | 1222 | 3669 | 72477 | 76145 | minus |
| | | Helicase | | | | | |
| odv-e28 | 1488710 | orf96 | 182 | 549 | 76132 | 76680 | plus |
| 38k | 1488713 | 38K | 320 | 963 | 78661 | 79623 | minus |
| lef-5 | 1488714 | LEF-5 | 265 | 798 | 79558 | 80355 | plus |
| 49K | 1488750 | orf142 | 476 | 1431 | 113396 | 114826 | plus |
| odv-e18 | 1488751 | ODV-E18 | 101 | 306 | 114834 | 115139 | plus |

The helicase is required for replication, lef5 is required for transcription and 38K, 49K and odv-e18 are required for viral structure, packaging and assembly.

The present inventors have identified further genes that are important for viral function. Accordingly, in a preferred embodiment the NPV alpha baculovirus clade Ia genome of the invention comprises at least one of the genes encoding lcf-2, lef-1, p47, lef-8 vp1054, lef-9, dnapo1, ac66, vlf-1, gp41, ac81, p95, capsid, lef-4, p33, p18, odv-c25, p6.9, odv-eo43, alk-exo, and odv-ec27. Preferably, the genome of the invention comprises all of these genes. These genes are indicated in Table 15a with reference to the genome of AcMNPV and are designated as vital genes of category II:

TABLE 15
AcMNPV

| Name | UniProt | Definition | aa | bp | Start | Stop | Strand |
|---|---|---|---|---|---|---|---|
| lef-2 | P41418 | late expression factor 2 | 210 | 633 | 3089 | 3721 | Plus |
| lef-1 | P41417 | late expression factor 2 | 266 | 801 | 10513 | 11313 | Minus |
| p47 | P34051 | transcription regulator | 401 | 1206 | 32177 | 33382 | Minus |
| lef-8 | P41452 | late expression factor 8 | 876 | 2631 | 40523 | 43153 | Minus |
| vp1054 | P41458 | viral capsid associated protein | 365 | 1098 | 45222 | 46319 | Plus |
| lef-9, | P41465 | late expression factor 9 | 516 | 1551 | 49184 | 50734 | Plus |
| Dnapol | P18131 | DNA-dependant DNA- | 984 | 2955 | 52329 | 55283 | Minus |
| | | polymerase | | | | | |
| ac66 | P41467 | AcOrf-66 peptide | 808 | 2427 | 55292 | 57718 | Plus |
| vlf-1 | Q06687 | very late expression | 379 | 1140 | 63813 | 64952 | Minus |
| | | factor 1 | | | | | |
| gp41 | P32651 | occlusion-derived virus | 409 | 1230 | 65607 | 66836 | Minus |
| | | glycoprotein | | | | | |
| ac81 | Q06694 | AcOrf-81 peptide | 233 | 702 | 66826 | 67527 | Minus |
| p95 | Q06670 | viral capsid associated protein | 847 | 2544 | 67884 | 70427 | Plus |
| Capsid | P17499 | major viral capsid protein | 347 | 1044 | 75534 | 76577 | Minus |
| lef-4 | P41477 | late expression factor 4 | 464 | 1395 | 76596 | 77990 | Plus |
| p33 | P41480 | AcOrf-92 peptide | 259 | 780 | 78699 | 79478 | Minus |
| p18 | P41481 | AcOrf-93 peptide | 161 | 486 | 79477 | 79962 | Plus |
| odv-e25 | P41483 | occlusion-derived virus | 228 | 687 | 79971 | 80657 | Plus |
| | | envelope protein | | | | | |
| p6.9 | P06545 | basic protein | 55 | 168 | 86712 | 86879 | Minus |
| Odv-ec43 | P41662 | AcOrf-109 peptide | 390 | 1173 | 94721 | 95893 | Minus |
| alk-exo | P24731 | alkaline exonuclease | 419 | 1260 | 112560 | 113819 | Plus |
| Odv-ec27 | P41702 | occlusion-derived virus | 290 | 873 | 125357 | 126229 | Plus |
| | | envelope/capsid protein | | | | | |

Accordingly, in a preferred embodiment the BmNPV genome of the invention comprises at least one of the genes encoding lef-1, pif-2, p47, lef-8, vp1054, lef-9, dnapo1, bm56 ac68, vlf-1, gp41, bm67 ac81, p95, capsid, lef-4, p33, p6.9, odv-ec43, pif-3, pif-1, alk-exo, p74, odv-ec27, odv-c56, and lef-2. Preferably, the genome of the invention comprises all of these genes. These genes are indicated in Table 15b with reference to the genome of BmNPV and are designated as vital genes of category II:

TABLE 15b
BmNPV

| Name | UniProt | Definition | aa | bp | Start | Stop | Strand |
|---|---|---|---|---|---|---|---|
| lef-1 | O92381 | LEF-1 | 270 | 813 | 5480 | 6292 | Minus |
| pif-2 | O92389 | AcMNPV | 382 | 1149 | 12335 | 13483 | Plus |
| | | orf22 | | | | | |
| p47 | O92407 | P47 | 399 | 1200 | 28266 | 29465 | Minus |
| lef-8 | O92415 | LEF-8 | 877 | 2634 | 35594 | 38227 | Minus |
| vp1054 | O92420 | VP1054 | 365 | 1098 | 40300 | 41397 | Plus |
| lef-9 | O92427 | LEF-9 | 490 | 1473 | 44402 | 45874 | Plus |
| dnapol | P41712 | DNA | 986 | 2961 | 47493 | 50453 | Minus |
| | | Polymerase | | | | | |
| bm56 | O92432 | AcMNPV | 134 | 405 | 54058 | 54462 | Plus |
| ac68 | | orf68 | | | | | |
| vlf-1 | O92440 | VLF-1 | 379 | 1140 | 58190 | 59329 | Minus |
| gp41 | O92443 | GP41/P40 | 403 | 1212 | 59987 | 61198 | Minus |
| bm67 | O92444 | AcMNPV | 234 | 705 | 61188 | 61892 | Minus |
| ac81 | | orf81 | | | | | |
| p95 | O92446 | P95 | 839 | 2520 | 62249 | 64768 | Plus |
| capsid | O92449 | VP39 | 350 | 1053 | 67520 | 68572 | Minus |
| lef-4 | O92450 | LEF-4 | 465 | 1398 | 68591 | 69988 | Plus |
| p33 | O92452 | AcMNPV | 259 | 780 | 70485 | 71264 | Minus |
| | | orf92 | | | | | |
| p6.9 | P24649 | DNA | 65 | 198 | 80352 | 80549 | Minus |
| | | Binding | | | | | |
| odv- | O92468 | AcMNPV | 391 | 1176 | 87784 | 88959 | Minus |
| ec43 | | orf109 | | | | | |
| pif-3 | O92472 | AcMNPV | 204 | 615 | 91385 | 91999 | Minus |
| | | orf115 | | | | | |
| pif-1 | O92475 | AcMNPV | 527 | 1584 | 92532 | 94115 | Plus |
| | | orf119 | | | | | |
| alk-exo | O92487 | ALK-EXO | 420 | 1263 | 104200 | 105462 | Plus |
| p74 | O92491 | P74 | 645 | 1938 | 108796 | 110733 | Minus |
| odv- | O92496 | ODV-EC27 | 290 | 873 | 115154 | 116026 | Plus |
| ec27 | | | | | | | |
| odv-e56 | O92500 | ODV-E56 | 375 | 1128 | 118837 | 119964 | Minus |
| lef-2 | O55457 | LEF-2 | 210 | 633 | 127652 | 128284 | Plus |

Lef-2, lef-1 and dna1 are required for DNA replication, p47, lef-8, lef-9 and lef-4 are required for transcription, vp1054, vlf-1, gp41, p95, capsid, p33, p6.9, odv-o43 and alk-exo are required for viral packaging and assembly, ac66, ac81, and odv-ec27 are required for host interaction and odv-e25 required for viral structure (ODV envelope).

The present inventors have identified further genes that are important for viral function. Accordingly, in a preferred embodiment the NPV alpha baculovirus clade Ia genome of the invention comprises at least one of the genes encoding pk-1, 38.7K, dbp, lef-6, ac29, 39K, lef-11, ac38, ac53, fp, lef-3, ac75, ac76, ac78 tlp20, p40, p12, p48, ac106/107 Ni, ac106/107 Ct, ac110, me53 and ie-1. Preferably, the genome of the invention comprises all of these genes. These genes are indicated in Table 16a with reference to the genome of AcMNPV and are designated as vital genes of category III:

TABLE 16a
AcMNPV

| Name | UniProt | Definition | aa | bp | Start | Stop | Strand |
|---|---|---|---|---|---|---|---|
| pk-1 | P41415 | protein kinase | 272 | 819 | 6917 | 7735 | Plus |
| 38.7K | P41423 | AcOrf-13 peptide | 327 | 984 | 9638 | 10621 | Minus |
| Dbp | P41430 | ssDNA binding protein | 316 | 951 | 21183 | 22133 | Minus |
| lef-6 | P41432 | late expression factor 6 | 173 | 522 | 23465 | 23986 | Plus |
| ac29 | P41433 | AcOrf-29 peptide | 71 | 216 | 24046 | 24261 | Minus |
| 39K | P11042 | nuclear matrix associated | 275 | 828 | 29242 | 30069 | Minus |
| | | phosphoprotein | | | | | |
| lef-11 | P21288 | late expression factor 11 | 112 | 339 | 30063 | 30401 | Minus |
| ac38 | P21290 | AcOrf-38 peptide | 216 | 651 | 30364 | 31014 | Minus |
| ac53 | P41457 | AcOrf-53 peptide | 139 | 420 | 44712 | 45131 | Plus |
| Fp | P69037 | Fp protein | 214 | 645 | 48513 | 49157 | Minus |
| lef-3 | P41453 | late expression factor 3 | 385 | 1158 | 57721 | 58878 | Minus |
| ac75 | Q06699 | AcOrf-75 peptide | 133 | 402 | 63126 | 63527 | Minus |
| ac76 | Q06690 | AcOrf-76 peptide | 84 | 255 | 63543 | 63797 | Minus |
| ac78 | Q06693 | AcOrf-78 peptide | 109 | 330 | 64958 | 65287 | Minus |
| tlp20 | Q06691 | telokin-like protein-20 | 180 | 543 | 67376 | 67918 | Minus |
| p40 | P25695 | hypothetical protein | 361 | 1086 | 86921 | 88006 | Minus |
| | | ACNVgp102 | | | | | |
| p12 | P41482 | AcOrf-102 | 122 | 369 | 88026 | 88394 | Minus |
| p48 | Q00732 | hypothetical protein | 387 | 1164 | 88375 | 89538 | Minus |
| | | ACNVgp104 | | | | | |
| ac106/107 Nt | P41659 | AcOrf-106 peptide | 61 | 186 | 93873 | 94058 | Plus |
| ac106/107 Ct, | P41660 | AcOrf-107 peptide | 110 | 333 | 94059 | 94391 | Plus |
| ac110, | P41663 | AcOrf-110 peptide | 56 | 171 | 95929 | 96099 | Minus |
| me53 | Qo4719 | DNA synthesis | 449 | 1350 | 121205 | 122554 | Minus |
| | | regulator | | | | | |
| ie-1 | P11138 | early gene transactivator | 582 | 1749 | 127198 | 128946 | Plus |

Accordingly, in a preferred embodiment the NPV alpha baculovirus clade Ia genome of the invention comprises at least one of the genes encoding polyhedrin, pk-1, 38.7K ac13, env-prot, dbp, lef-6, bm20 ac29, v-ubi, 39k, lef-11, bm29 ac38, bm42 ac53, fp, bm54 ac66, lef-3, bm61 ac75, bm62 ac76, bm64 ac78, tlp20, p18, odv-e25, p40, p12, p45 Ac p48, bm90 ac106/107, bm92a ac110, me53, bm121 ac145, bm122 ac146 and io-1. Preferably, the genome of the invention comprises all of these genes. These genes are indicated in Table 16b with reference to the genome of BmNPV and are designated as vital genes of category III:

TABLE 16b
BmNPV

| Name | UniProt | Definition | aa | bp | Start | Stop | Strand |
|---|---|---|---|---|---|---|---|
| polyhedrin | 1724487 | Polyhedrin | 245 | 738 | 1 | 738 | Plus |
| pk-1 | 1724485 | Protein Kinase | 275 | 828 | 2395 | 3222 | Plus |
| 38.7K ac13 | 1488635 | AcMNPV orf13 | 331 | 996 | 4605 | 5600 | Minus |
| env-prot | 1488645 | AcMNPV orf23 | 673 | 2022 | 13586 | 15607 | Plus |
| dbp | 1488647 | DBP | 317 | 954 | 16186 | 17139 | Minus |
| lef-6 | 1488650 | LEF-6 | 173 | 522 | 18489 | 19010 | Plus |
| bm20 ac29 | 1488651 | AcMNPV orf29 | 71 | 216 | 19129 | 19344 | Minus |
| v-ubi | 1724483 | Ubiquitin | 77 | 234 | 25035 | 25268 | Plus |
| 39k | 1488658 | 39K | 277 | 834 | 25321 | 26154 | Minus |
| lef-11 | 1488659 | LEF-11 | 112 | 339 | 26148 | 26486 | Minus |
| bm29 ac38 | 1488660 | AcMNPV orf38 | 217 | 654 | 26449 | 27102 | Minus |
| bm42 ac53 | 1488673 | AcMNPV orf53 | 139 | 420 | 39790 | 40209 | Plus |
| fp | 1488681 | 25K | 214 | 645 | 43654 | 44298 | Minus |
| bm54 ac66 | 1488685 | AcMNPV orf66 | 805 | 2418 | 50462 | 52879 | Plus |
| lef-3 | 1488686 | LEF-3 | 385 | 1158 | 52882 | 54039 | Minus |
| bm61 ac75 | 1488693 | AcMNPV orf75 | 133 | 402 | 57497 | 57898 | Minus |
| bm62 ac76 | 1488694 | AcMNPV orf76 | 85 | 258 | 57917 | 58174 | Minus |
| bm64 ac78 | 1488696 | AcMNPV orf78 | 110 | 333 | 59335 | 59667 | Minus |
| tlp20 | 1488700 | AcMNPV orf82 | 181 | 546 | 61738 | 62283 | Minus |
| p18 | 1488708 | AcMNPV orf93 | 161 | 486 | 71263 | 71748 | Plus |
| odv-e25 | 1488709 | ODV-E25 | 228 | 687 | 71757 | 72443 | Plus |
| p40 | 1488715 | AcMNPV orf101 | 362 | 1089 | 80591 | 81679 | Minus |
| p12 | 1488716 | AcMNPV orf102 | 123 | 372 | 81699 | 82070 | Minus |
| p45 Ac p48 | 1488717 | AcMNPV orf103 | 387 | 1164 | 82051 | 83214 | Minus |
| bm90 ac106/107 | 1488720 | AcMNPV orf106 | 249 | 750 | 86702 | 87451 | Plus |
| bm92a ac110 | 1488723 | AcMNPV orf110 | 59 | 180 | 88983 | 89162 | Minus |
| me53 | 1488748 | ME53 | 451 | 1356 | 110964 | 112319 | Minus |
| bm121 ac145 | 1488753 | AcMNPV orf145 | 95 | 288 | 116041 | 116328 | Plus |
| bm122 ac146 | 1488754 | AcMNPV orf146 | 201 | 606 | 116323 | 116928 | Minus |
| ie-1 | 1488755 | IE-1 | 584 | 1755 | 116994 | 118748 | Plus |

Dbp, lef-11, ac38, lef-3, me53 and ic-1 are required for replication, 38.7K, lef-6 and 39K are required for transcription, fp, ac75, p40 required for virus structure, pk-1, ac53 and p12 are required for host interaction and ac29 ac76, ac78, tlp20, p48, ac106/107 Nt, ac106/107 Ct, and ac110 are of unknown function.

The present inventors have identified further genes that are important for viral function. Accordingly, in a preferred embodiment the NPV alpha baculovirus clade Ia genome of the invention comprises at least one of the genes encoding ac12, ac34, ac55, and ac108. Preferably, the genome of the invention comprises all of these genes. These genes are indicated in Table 17a with reference to the genome of AcMNPV and are designated as vital genes of category IV:

TABLE 17a
AcMNPV

| Name | UniProt | Definition | aa | bp | Start | Stop | Strand |
|---|---|---|---|---|---|---|---|
| ac12 | P41422 | AcOrf-12 peptide | 217 | 654 | 8958 | 9611 | Plus |
| ac34 | P21287 | AcOrf-34 peptide | 215 | 648 | 28294 | 28941 | Minus |
| ac55 | P41459 | AcOrf-55 peptide | 73 | 222 | 46411 | 46632 | Plus |
| ac108 | P41661 | AcOrf-108 peptide | 105 | 318 | 94392 | 94709 | Minus |

Accordingly, in a preferred embodiment the NPV alpha baculovirus clade Ia genome of the invention comprises at least one of the genes encoding egt, bm25 ac34, lef-10, bm44 ac55, chaB, bm48 ac60, vp80, bm91 ac108, p24, pp34 and ic-0. Preferably, the genome of the invention comprises all of these genes. These genes are indicated in Table 17b with reference to the genome of BmNPV and are designated as vital genes of category IV:

TABLE 17b
BmNPV

| Name | UniProt | Definition | aa | bp | Start | Stop | Strand |
|---|---|---|---|---|---|---|---|
| egt | 1488637 | UDP-Glucosyl Transferase | 506 | 1521 | 6407 | 7927 | Plus |
| bm25 ac34 | 1488657 | AcMNPV orf34 | 215 | 648 | 24367 | 25014 | Minus |
| lef-10 | 1488674 | LEF-10 | 78 | 237 | 40206 | 40442 | Plus |
| bm44 ac55 | 1488676 | AcMNPV orf55 | 77 | 234 | 41479 | 41712 | Plus |
| chaB | 1488679 | AcMNPV orf58 | 171 | 516 | 42723 | 43238 | Minus |
| bm48 ac60 | 1488680 | AcMNPV orf60 | 83 | 252 | 43250 | 43501 | Minus |
| vp80 | 1488718 | VP80 | 692 | 2079 | 83240 | 85318 | Plus |
| bm91 ac108 | 1488721 | AcMNPV orf108 | 105 | 318 | 87452 | 87769 | Minus |
| p24 | 1488737 | P24 | 195 | 588 | 101563 | 102150 | Plus |
| pp34 | 1488739 | PP34 | 315 | 948 | 102560 | 103507 | Plus |
| ie-0 | 1488749 | IE-0 | 261 | 786 | 112596 | 113381 | Plus |

These genes are of unknown function.

The present inventors have identified further genes that are important for viral function.

Accordingly, in a preferred embodiment the NPV alpha baculovirus clade Ia genome of the invention comprises at least one of the genes encoding ac4, ac5, orf1629, ac17+45, ac19, arif-1 Ct, arif-1 Nt, pkip, ac26, lcf-12, ao43, ac48, bjdp, ac72, ac73, ac74, ac79, ac111, ac114, ac120, ac124, lcf-7, gp67, gp16, ac132, ie-2, pe38. Preferably, the genome of the invention comprises all of these genes. These genes are indicated in Table 18a with reference to the genome of AcMNPV and are designated as vital genes of category V:

TABLE 18a
AcMNPV

| Name | UniProt | Definition | aa | bp | Start | Stop | Strand |
|---|---|---|---|---|---|---|---|
| ac4 | P25654 | AcOrf-4 peptide | 151 | 456 | 2295 | 2750 | Minus |
| ac5 | P41420 | AcOrf-5 peptide | 109 | 330 | 2779 | 3108 | Plus |
| orf1629 | Q03209 | viral capsid associated, protein | 543 | 1632 | 5287 | 6918 | Minus |
| ac17 + 45 | P12826 | AcOrf-17 peptide | 164 | 495 | 13738 | 14232 | Plus |
| ac19 | P41424 | AcOrf-19 peptide | 108 | 327 | 15461 | 15787 | Plus |
| arif-1 Ct | P41425 | actin rearrangement inducing | 69 | 210 | 16013 | 16222 | Minus |
| | | factor | | | | | |
| arif-1 Nt | P41426 | actin rearrangement | 319 | 960 | 16305 | 17264 | Minus |
| | | inducing-factor | | | | | |
| pkip | P41429 | protein kinase interacting | 169 | 510 | 20634 | 21143 | Minus |
| | | protein | | | | | |
| ac26 | P41431 | AcOrf-26 peptide | 129 | 390 | 22209 | 22598 | Plus |
| lef-12 | P41446 | AcOrf-41 peptide, late | 181 | 546 | 33381 | 33926 | Plus |
| | | expression factor 12 | | | | | |
| ac43 | P41448 | AcOrf-43 peptide | 77 | 234 | 35544 | 35777 | Plus |
| ac48 | P11039 | AcOrf-48 peptide | 113 | 342 | 39278 | 39619 | Minus |
| bjdp | P41455 | AcOrf-51 peptide | 318 | 957 | 43180 | 44136 | Plus |
| ac72 | P41471 | AcOrf-72 peptide | 60 | 183 | 61824 | 62006 | Plus |
| ac73 | P41472 | AcOrf-73 peptide | 99 | 300 | 62015 | 62314 | Minus |
| ac74 | P41473 | AcOrf-74 peptide | 265 | 798 | 62311 | 63108 | Minus |
| ac79 | Q06692 | AcOrf-79 peptide | 104 | 315 | 65290 | 65604 | Minus |
| ac111 | P41664 | AcOrf-111 peptide | 67 | 204 | 96148 | 96351 | Minus |
| ac114 | P41667 | AcOrr-114 peptide | 424 | 1275 | 97886 | 99160 | Minus |
| ac120 | P41673 | AcOrf-120 peptide | 82 | 249 | 102296 | 102544 | Plus |
| ac124 | P41679 | AcOrf-124 peptide | 247 | 744 | 103793 | 104536 | Plus |
| lef-7 | P41677 | late expression factor 7 | 226 | 681 | 104553 | 105233 | Minus |
| gp67 | P17501 | major budded virus | 512 | 1539 | 108179 | 109717 | Minus |
| | | envelope glycoprotein | | | | | |
| gp16 | P24729 | hypothetical protein | 106 | 321 | 110524 | 110844 | Plus |
| | | ACNVgp131 | | | | | |
| ac132 | P24730 | AcOrf-132 peptide | 219 | 660 | 111873 | 112532 | Plus |
| ie-2 | P24647 | early gene transactivator | 408 | 1227 | 130857 | 132083 | Minus |
| pe38 | P23801 | hypothetical protein | 321 | 966 | 132526 | 133491 | Plus |
| | | ACNVgp155 | | | | | |

The present inventors have identified further genes that are important for viral function. Accordingly, in a preferred embodiment the NPV alpha baculovirus clade Ia genome of the invention comprises at least one of the genes and, preferably, the genome of the invention comprises all of these genes orf1629, bm4 ac11, bv/odv-e26, bm9 ac17, bm10 ac18, bm11 ac19, arif-1, pkip, bm17 ac26, iap-1, bm21 ac30, lef-12, bm34 ac43, bjdp, gp37, iap-2, bm58a ac72, bm59 ac73, bm60 ac74, bm65 ac79, bm93 ac111, bm94 ac114, bm96, bm98 ac120, bm101 ac124, lef-7, gp64/67, p116, bm109 ac132, p26, p10, io-2, pe38, ptp, bm133 ac4 and bm134 ac5. These genes are indicated in Table 18b with reference to the genome of BmNPV and are designated as vital genes of category V:

TABLE 18b
BmNPV

| Name | UniProt | Definition | aa | bp | Start | Stop | Strand |
|---|---|---|---|---|---|---|---|
| orf1629 | 1488633 | Orf1629 | 542 | 1629 | 768 | 2396 | minus |
| bm4 ac11 | 1488634 | AcMNPV orf11 | 340 | 1023 | 3248 | 4270 | minus |
| bv/odv-e26 | 1488639 | BV/ODV-E26 | 229 | 690 | 8067 | 8756 | plus |
| bm9 ac17 | 1488640 | AcMNPV orf17 | 210 | 633 | 8725 | 9357 | plus |
| bm10 ac18 | 1488641 | AcMNPV orf18 | 356 | 1071 | 9387 | 10457 | minus |
| bm11 ac19 | 1488642 | AcMNPV orf19 | 110 | 333 | 10459 | 10791 | plus |
| arif-1 | 1488643 | ARIF-1 | 440 | 1323 | 10976 | 12298 | minus |
| pkip | 1488646 | PKIP | 169 | 510 | 15637 | 16146 | minus |
| bm17 ac26 | 1488648 | AcMNPV orf26 | 129 | 390 | 17215 | 17604 | plus |
| iap-1 | 1488649 | IAP1 | 292 | 879 | 17606 | 18484 | plus |
| bm21 ac30 | 1488652 | AcMNPV orf30 | 472 | 1419 | 19399 | 20817 | minus |
| lef-12 | 1488663 | AcMNPV orf41 | 183 | 552 | 29525 | 30076 | plus |
| bm34 ac43 | 1488665 | orf43-like protein | 78 | 237 | 31686 | 31922 | plus |
| bjdp | 1488671 | AcMNPV orf51 | 319 | 960 | 38254 | 39213 | plus |
| gp37 | 1488684 | GP37 | 294 | 885 | 46479 | 47363 | minus |
| iap-2 | 1488689 | IAP2 | 249 | 750 | 55377 | 56126 | plus |
| bm58a ac72 | 1488690 | AcMNPV orf72 | 60 | 183 | 56185 | 56367 | plus |
| bm59 ac73 | 1488691 | AcMNPV orf73 | 99 | 300 | 56377 | 56676 | minus |
| bm60 ac74 | 1488692 | AcMNPV orf74 | 268 | 807 | 56673 | 57479 | minus |
| bm65 ac79 | 1488697 | AcMNPV orf79 | 104 | 315 | 59670 | 59984 | minus |
| bm93 ac111 | 1488724 | AcMNPV orf111 | 67 | 204 | 89211 | 89414 | minus |
| bm94 ac114 | 1488725 | AcMNPV orf114 | 424 | 1275 | 90089 | 91363 | minus |
| bm98 ac120 | 1488730 | AcMNPV orf120 | 82 | 249 | 94123 | 94371 | plus |
| bm101 ac124 | 1488734 | AcMNPV orf124 | 244 | 735 | 95620 | 96354 | plus |
| lef-7 | 1488735 | LEF-7 | 227 | 684 | 96376 | 97059 | minus |
| gp64/67 | 1488736 | GP64/67 | 530 | 1593 | 99844 | 101436 | minus |
| gp16 | 1488738 | GP16 | 106 | 321 | 102178 | 102498 | plus |
| bm109 ac132 | 1488740 | AcMNPV orf132 | 220 | 663 | 103510 | 104172 | plus |
| p26 | 1488745 | P26 | 240 | 723 | 107702 | 108424 | plus |
| p10 | 1488746 | P10 | 70 | 213 | 108497 | 108709 | plus |
| ie-2 | 1488759 | IE-2 | 422 | 1269 | 120662 | 121930 | minus |
| pe38 | 1488760 | PE38 | 309 | 930 | 122416 | 123345 | plus |
| ptp | 1488762 | PTP | 168 | 507 | 124431 | 124937 | plus |
| bm133 ac4 | 1488765 | AcMNPV orf4 | 151 | 456 | 126858 | 127313 | minus |
| bm134 ac5 | 1488766 | orf5-like protein | 109 | 330 | 127342 | 127671 | plus |

Ac79 and lef-7 are required for replication, lef-12 and bjdp are required for transcription, orf1629 is required for virus structure ao4, arif-1 Ct/Nt, pkip, gp67, ie-2 and pe38 are required for host interaction and ac5, ac17+45, ac19, ac26, ac43, ac48, ac72, ac73, ac74, ac111, ac114, ac120, ac124, gp16, and ac132 are of unknown.

The present inventors have identified further genes that are important for viral function. Accordingly, in a preferred embodiment the NPV alpha baculovirus clade Ia genome of the invention comprises at least one of the genes encoding ac45, ao47, ac52+71, he65, 35K and ac154. Preferably, the genome of the invention comprises all of these genes. These genes are indicated in Table 19a with reference to the genome of AcMNPV and are designated as vital genes of category VI:

TABLE 19a
AcMNPV

| Name | UniProt | Definition | aa | bp | Start | Stop | Strand |
|---|---|---|---|---|---|---|---|
| ac45 | P41450 | AcOrf-45 peptide | 192 | 579 | 36155 | 36733 | Plus |
| ac47 | P11040 | AcOrf-47 peptide | 88 | 267 | 38938 | 39204 | Minus |
| ac52 +71 | P41456 | AcOrf-52 peptide | 123 | 372 | 44339 | 44710 | Minus |
| he65 | Q08539 | hypothetical protein ACNVgp106 | 553 | 1662 | 91667 | 93328 | Minus |
| 35K | P08160 | annihilator | 299 | 900 | 116492 | 117391 | Plus |
| ac154 | P41709 | AcOrf-154 peptide | 81 | 246 | 133591 | 133836 | Plus |

These genes are indicated in Table 19b with reference to the genome of BmNPV and are designated as vital genes of category VI:

TABLE 19b
BmNPV

| Name | UniProt | Definition | aa | bp | Start | Stop | Strand |
|---|---|---|---|---|---|---|---|
| orf1629 | 1488633 | Orf1629 | 542 | 1629 | 768 | 2396 | minus |
| bm4 ac11 | 1488634 | AcMNPV orf11 | 340 | 1023 | 3248 | 4270 | minus |
| bm9 ac17 | 1488640 | AcMNPV orf17 | 210 | 633 | 8725 | 9357 | plus |
| bm10 ac18 | 1488641 | AcMNPV orf18 | 356 | 1071 | 9387 | 10457 | minus |
| bm11 ac19 | 1488642 | AcMNPV orf19 | 110 | 333 | 10459 | 10791 | plus |
| arif-1 | 1488643 | ARIF-1 | 440 | 1323 | 10976 | 12298 | minus |
| pkip | 1488646 | PKIP | 169 | 510 | 15637 | 16146 | minus |
| bm17 ac26 | 1488648 | AcMNPV orf26 | 129 | 390 | 17215 | 17604 | plus |
| iap-1 | 1488649 | IAP1 | 292 | 879 | 17606 | 18484 | plus |
| sod | 1488655 | SOD | 151 | 456 | 22029 | 22484 | plus |
| p43 | 1488661 | P43 | 362 | 1089 | 27170 | 28258 | minus |
| lef-12 | 1488663 | AcMNPV orf41 | 183 | 552 | 29525 | 30076 | plus |
| bm34 ac43 | 1488665 | orf43-like protein | 78 | 237 | 31686 | 31922 | plus |
| bm35 ac44 | 1488666 | AcMNPV orf44 | 131 | 396 | 31903 | 32298 | plus |
| bm36 ac45 | 1488667 | AcMNPV orf45 | 193 | 582 | 32300 | 32881 | plus |
| ets ac47 | 1488669 | ETS | 89 | 270 | 35073 | 35342 | minus |
| bjdp | 1488671 | AcMNPV orf51 | 319 | 960 | 38254 | 39213 | plus |
| bm41 ac52 | 1488672 | AcMNPV orf52 | 194 | 585 | 39204 | 39788 | minus |
| bm45 ac56 | 1488677 | AcMNPV orf56 | 84 | 255 | 41714 | 41968 | plus |
| bm51 ac63 | 1488683 | AcMNPV orf63 | 155 | 468 | 45935 | 46402 | plus |
| gp37 | 1488684 | GP37 | 294 | 885 | 46479 | 47363 | minus |
| bm57 ac69 | 1488688 | AcMNPV orf69 | 262 | 789 | 54440 | 55228 | plus |
| iap-2 | 1488689 | IAP2 | 249 | 750 | 55377 | 56126 | plus |
| bm60 ac74 | 1488692 | AcMNPV orf74 | 268 | 807 | 56673 | 57479 | minus |
| bm65 ac79 | 1488697 | AcMNPV orf79 | 104 | 315 | 59670 | 59984 | minus |
| he65 | 1488719 | HE65 | 289 | 870 | 85343 | 86212 | minus |
| bm93 ac111 | 1488724 | AcMNPV orf111 | 67 | 204 | 89211 | 89414 | minus |
| bm98 ac120 | 1488730 | AcMNPV orf120 | 82 | 249 | 94123 | 94371 | plus |
| lef-7 | 1488735 | LEF-7 | 227 | 684 | 96376 | 97059 | minus |
| chi-a | 1724489 | CHITINASE | 552 | 1659 | 97049 | 98707 | minus |
| v-cath | 1724490 | Cystein Protease | 323 | 972 | 98756 | 99727 | plus |
| gp16 | 1488738 | GP16 | 106 | 321 | 102178 | 102498 | plus |
| p35 | 1488744 | P35 | 299 | 900 | 105923 | 106822 | plus |
| p26 | 1488745 | P26 | 240 | 723 | 107702 | 108424 | plus |
| p10 | 1488746 | P10 | 70 | 213 | 108497 | 108709 | plus |
| bm125 ac149 | 1488757 | AcMNPV orf149 | 106 | 321 | 119993 | 120313 | minus |
| bm126 ac150 | 1488758 | AcMNPV orf150 | 115 | 348 | 120282 | 120629 | plus |
| pe38 | 1488760 | PE38 | 309 | 930 | 122416 | 123345 | plus |
| bm129 ac154 | 1488761 | AcMNPV orf154 | 77 | 234 | 123446 | 123679 | plus |
| bm133 ac4 | 1488765 | AcMNPV orf4 | 151 | 456 | 126858 | 127313 | minus |

ac52+71 and he65 appear required for replication, 35K appears required for host interaction and ac45, ao47, and he65 are of unknown function.

It is preferred that the genome of the present invention comprises one or all, preferably all of the following genes: (i) vital genes of category I; (ii) vital genes of category II; (iii) vital genes of category III; (iv) vital genes of category IV; (v) vital genes of category V: (vi) vital genes of category VI; (vii) vital genes of category I and II; (viii) vital genes of category I and III; (ix) vital genes of category I and IV; (x) vital genes of category I and V; (xi) vital genes of category I and VI; (xii) vital genes of category I, II and m; (xiii) vital genes of category I, II and IV; (xiv) vital genes of category I, II and V; (xv) vital genes of category I, II and VI; (xvi) vital genes of category I, III and IV; (xvii) vital genes of category I, III and V; (xviii) vital genes of category I, III and VI; (xix) vital genes of category I, IV and V; (xx) vital genes of category I, IV and VI; (xxi) vital genes of category I, V and VI; (xxii) vital genes of category I, II, II and IV; (xxiii) vital genes of category I, II, III and V; (xxiv) vital genes of category I, II, III and VI; (xxv) vital genes of category I, II, IV and V; (xxvi) vital genes of category I, II, IV and VI; (xvii) vital genes of category I, II, V and VI; (xxix) vital genes of category I, II, III, IV and V; (xxx) vital genes of category I, II, III, IV and VI; (xxxi) vital genes of category I, III, IV, V and VI; (xxxii) vital genes of category I, II, IV, V and VI; (xxxii) vital genes of category I, II, III, V and VI; (xxxiii) vital genes of category I, II, III, IV, V and VI.

In each of above cases it is preferred that the genome comprises both the CDS as well as any 5′-UTR and/or 3′-UTR flanking the CDS.

The present inventors also found that the following CDSs of the hr4-5 section (bp 99182-121072) can be deleted completely without detrimental effect on AcMNPV: p26, p10, p74, pif-3, ac116, ac117, ac118, ac121, ac122, pk-2, v-cath, pp34 and/or 94K. Also, the CDSs of the genes pif-1 and chiA of the hr4-5 section can be deleted partially (truncated) without detrimental effect on AcMNPV. This is because these genes are not essential for virus infection and propagation, but since the promoter regions of adjacent genes overlap with their CDSs, a portion of pif-1 and chiA has to remain. For pif-1, the portion that may be deleted is bp 100699 to bp102199 of the AcMNPV genome (pif-1 itself extends from bp 100699 to bp 102291 of the AcMNPV genome), and for chiA, this portion is bp 105560 to bp 106937 of the AcMNPV genome (chiA itself extends from bp 105282 to bp 106937 of the AcMNPV genome). In other words, the portion that must remain of pif-1 in the genome is bp 102200 to bp 102291 of the AcMNPV genome and the portion that must remain of chiA in the genome is bp 105282 to bp 105559 of the AcMNPV genome. Accordingly, pif-1 and chiA are partially deleted such that the promoter regions of the adjacent genes remain. This applies to all embodiments mentioned herein, which relate to pif-1 and/or chiA. In a further general embodiment, genes taught to be deletable herein may be deleted only in as far as portions of the sequence remain which are part of the CDS or the 5′/3′ UTR of a gene taught to be essential.

Thus, in a further embodiment, the NPV alpha baculovirus clade Ia genome according to the first aspect of the invention lacks at least one, two, three, four, five, six, seven, eight, nine, ten, eleven, twelve, thirteen or fourteen genes, preferably all genes of the group consisting of pif-1, p26, p10, p74, pif-3, ac116, ac117, ac118, ac121, ac122, pk-2, chiA, v-cath, pp34 and 94K, wherein pif-1 and chiA are partially deleted such that the promoter regions of the adjacent genes remain. The adjacent genes are ac120 for pif-1 and lef-7 for chiA and the promoter region of these genes that overlap with pif-1 and chiA, respectively, are defined above. Preferably, said genome also lacks (i) the 5′-L TR and/or 3′-UTR (as defined above) and/or (ii) the spacers 5′ and/or 3′ (as defined above) of at least one, two, three, four, five, six, seven, eight, nine, ten, eleven, twelve, thirteen, fourteen or preferably all of these genes The present inventors also found that the following CDSs of the hr4-5 section (bp 99182-121072) are important to maintain vital functions of AcMNPV: ac120, ac124, lef-7, gp64/67, p24, gp16, ac132, alk-exo, and 35K. Thus, in a further embodiment, the NPV alpha baculovirus clade Ia genome according to the first aspect of the invention comprises at least one, two, three, four, five, six, seven, eight preferably all genes of the group consisting of ac120, ac124, lef-7, gp64/67, p24, gp16, ac132, alk-exo, and 35K. Preferably, said genome comprises also (i) the 5′-UTR and/or 3′-UTR (as defined above) and/or (ii) the spacers 5′ and/or 3′ (as defined above) of at least one, two, three, four, five, six, seven, eight, or preferably all of these genes.

Thus, in a preferred embodiment, the NPV alpha baculovirus clade Ia genome according to the first aspect of the invention (a) lacks at least one, two, three, four, five, six, seven, eight, nine, ten, eleven, twelve, thirteen or fourteen genes, preferably all genes of the group consisting of pif-1, p26, p10, p74, pif-3, ac116, ac1117, ac118, ac121, ac122, pk-2, chiA, v-cath, pp34 and 94K, wherein pif-1 and chiA are only partially deleted such that the promoter regions of the adjacent genes remain; and (b) comprises at least one, two, three, four, five, six, seven, eight or preferably all genes of the group consisting of ac120, ac124, lcf-7, gp64/67, p24, gp16, ac132, alk-exo, and 35K. Preferably, said genome lacks/comprises also (i) the 5′-UTR and/or 3′-UTR and/or (ii) the spacers 5′ and/or 3′ of at least 1, 2, 3, 4, 5, 6, 7, 8, 9, 10, 11, 12, 13, 14, 15, 16, 16, 17, 19, 20, 21, 22, 23 or preferably all of these genes.

In a second aspect the present invention provides a NPV alpha baculovirus clade Ia genome according to the first aspect of the invention further comprising a nucleotide sequence heterologous to the NPV alpha baculovirus clade Ia genome. The term “heterologous” in this context refers to nucleotide sequence not natively occurring in the genome of the respective NPV alpha baculovirus clade Ia genome or not occurring at that position. Accordingly, a native promoter of that baculovirus that is rearranged within the genome is also considered heterologous. Preferably, the term refers to nucleotide sequence not natively comprised in a baculovirus, more preferably a eukaryotic gene or cDNA. Particularly, preferred the gene or cDNA is of mammalian, e.g. mouse, rat, rabbit, dog, cat, human origin. It is particularly preferred that the heterologous nucleotide sequence comprises more than one gene. Due to the large size reduction the genome of the present invention can accommodate large sections of heterologous nucleotide sequences. The inserts can be as big as the native sequence removed. Accordingly, Table 9 can serve as an indication of the size of the heterologous nucleotide sequence that can be present in the genome of the invention. The expression of the genes comprised in the heterologous nucleotide sequence is preferably driven by IE1 or polyhedrin or p10 promoter.

Other preferred heterologous nucleotide sequence that may be comprised in the genome are a Tn7 site, a nucleotide sequence encoding a resistance gene, a Homing endonuclease site, a mutated fp gene, loxP sites, IE1/polyhedrin/p10 promoter, a nucleotide sequence encoding a fluorescent protein. Preferred fluorescent proteins comprise green, red or yellow fluorescent proteins or mCherry.

In a third aspect the present invention provides an infectious NPV alpha baculovirus clade Ia virus comprising a genome according to the first or the second aspect of the invention.

Using improved methodology reported by Smith and colleagues (Smith H O et al PNAS 2003) on accurate assembly of ˜10 Kb DNA segments, we aim to assemble our computationally identified assembled gene segments from synthetic long polynucleotide fragments using molecular biology techniques for DNA assembly (Stemmer W P C et al., Gene 1995, Gibson et al., Nature Methods 2009). PCR amplification would then be used to obtain large amounts of pure full-length genomes (15-Kb DNA segments) and finally, gel electrophoresis will be used to purify amplified gene segments. Having engineered and amplified the gene segment of interest, the wild-type sequence with this synthetic DNA fragment could be replaced in the current “wild-type” baculoviral genome (which itself has been already engineered by classical means). In principle, the synthetic gene segment could be inserted into baculoviruses using E. coli/yeast based recombination and in-vitro ligation (Zhao Y. et al., Nucleic Acids Res 2003). As reported by Gibson and colleagues on synthetic genome assembly strategy in yeast (Gibson D G et al., Science 2010), three stage of genome assembly using transformation and homologues recombination in yeast. As a first stage, 10 kb synthetic gene segments and vector would be recombined in yeast/E. coli and transferred to E. coli. Then, the vector with assembled segments will be isolated for positive selection. At the second and third stage, the multiple 10 kb fragments and wild-type genome fragments would be transformed in the yeast/E. coli which would produce larger second-stage and final stage assembly intermediate. Here in order to generate semi-synthetic genome assembly, yeast/E. coli would be co-transformed with the baculovirus wild-type gene fragments and PCR amplified vector with overlapping ends of the synthetic inserts.

Once the hybrid genome is constructed and isolated from the yeast, it will be characterized by screening with multiplex PCR. Further for comparison, natural genome extracted from the yeast/E. coli and baculovirus (wild-type) would be used for characterization analysis. Once characterized, semi-synthetic genome transplantation strategy will be used wherein semi-synthetic genome from yeast clones would be transplanted into recipient cells as described before (Benders G A et al, Nucleic acids Res. 2010). Further the functionality of the hybrid virus with the synthetic gene segment would be validated based on its self-replicating properties and also by expressing test proteins and complexes.

In a fourth aspect the present invention provides a cell infected with a virus according to the third aspect of the present invention. A large number of suitable cells are publically available (see, e.g. Lynn, D. E. (2007) Methods in Molecular Biology. Vol 338, Chapter 6 Baculovirus and Insect Cell Expression Protocols, Editor: Murhammer D. W., Humanan Press Inc.). Preferably, the cell is selected from the group consisting of Ao/I, Hi5, Sf9, Sf21, Ao38, Drosophila $2, T.ni, FTRS-AoL1/-AoL2/-AfL, BCIRL/AMCY-AiOV-CLG, BCIRL/AMCY-AiTS-CLG, HCRL-ATO10/ATO20, BCIRL/AMCY-AfOV-CLG, BCIRL/AMCY-AfTS-CLG, RML-2, NISES-AnPe-426, NISES-Anya-0611, BCIRL/AMCY-AgE-CLG-1/2/3, UFL-AG-286, FTRS-AbL81, SES-Bma-O1A/R, Bm-N/-5/-21E-HNU5, NIV-BM-1296/-197, SES-Bm-130A/30R/e21A/c 21B/e 21R, SES-BoMo-15A/-C129/-JI25, SPC-Bm36/-Bm40, WIV-BS-481/484, FPMI-CF-1/2/3, FPMI-CF-203, FPMI-CF-50/60/70, IPRI-CF-1/10/12, IPRI-Cf124, IPRI-CF-16/-16T, IPRI-CF-5/-6/-8, CP-1268, CP-169, CpDW1-15, IZD-Cp 4/13, IZD-CP1508/-CP2202/-CP2507/-CP0508, SIE-EO-801/-803, IPLB-Ekx4T/-Ekx4V, EA1174A, EA1174H, IAFEs-1, BCIRL-HA-AM1, CSIRO-BCIRL-HA1-3, CSIRO-BCIRL-HP1-5, BCIRL/AMCY-HzE-CLG1-9, BCIRL-HZ-AM1-3, IMC-HZ-1, IPLB-HZ-1074-5, IPLB-HZ-1079, IPLB-HZ-110, IPLB-HZ-124Q, BCIRL/AMCY-HvE-CLG1-3, BCIRL/AMCY-HvOV-CLG, BCIRL/AMCY-Hv-TS-GES, BCIRL-HV-AM1-2, IPLB-HvE1a/-It, IPLB-HvE1s, IPLB-HvE6a/-It, IPLB-HvE6s/-It, IPLB-HvT1, FTRS-HmA45, FTRS-HIL1-2, NIAS-LeSe-11 IPLB-LD-64-67, IPLB-LdEG/-LdEI/-LdEIt/-LdEp/-LdFB, IZD-LD1307/-LD1407, UMN-MDH-1, HPB-MB, IZD-MB0503/MB0504/MB1203/MB2006/2007/2506, MB-H260, MbL-3, NIAS-MaBr-85192/93, NIAS-MaBr-92, NIAS-MB-19/25/32, SES-MaBr-1/2/3/4/5, FPMI-MS-12/4/5/7, MRRL-CH-1/2, BPMNU-MyCo-1, IPLB-OE505A/s, IPLB-O1E7, IPRI-OL-12/13/4/9, BCIRL/AMCY-OnFB-GES1/2, UMC-OnE, FTRS-PhL, Px-58/-64, ORS-Pop-93/-95, BTI-PR10B/-PR8A1/-PR8A2/-PR9A, NIAS-PRC-819A/-819B/-819C, NYAES-PR4A, IAL-PID2, IPLB-PiE, UMN-PIE-1181, BCIRL/AMCY-PxLP-CLG, IPLB-PxE1/-PxE2, PX-1187, BCIRL-PX2-HNU3, BTI-Pu-2, BTI-Pu-A7/-A7S, BTI-Pu-B9, BTI-Pu-M, BTI-Pu-MIB, FRI-SpIm-1229, BCIRL/AMCY-ScE-CLG1/-CLG4/-CLG5, Se3FH, Se4FH, Se5FH, Se6FHA, Se6FHB, SeHe920-1a, UCR-SE-1, BCIRL/AMCY-SfTS-GES, IAL-SFD1, Sf1254, IPLB-Sf21AE, HPB-SL, SPC-SI-48/-52, UIV-SL-373/-573/-673, IBL-SLIA, NIV-SU-893/-992, BCIRL-503-HNU1/504-HNU4, BCIRL/AMCY-TnE-CLG1/-TnE-CLG1 MK, BCIRL/AMCY-TnE-CLG2/-TnE-LG2MK/-TnE-CLG3/-TnTS-GES1/-TnTS-GES3, BTI-TN5B1-4/-TN5C1/-TN5F2/-TN5G2A1/BTI-TN5G3/-TN5G33, IAL-TND1, IPLB-TN-R, and TN-368.

In a fifth aspect the present invention provides a method for producing an NPV alpha baculovirus clade Ia genome according to the first or second aspect of the invention comprising the step of chemically synthesizing all or part of the genome.

In this it is preferred that the part of the genome that flanks the regions that are deleted from the genome is synthesized. These parts are preferably inserted into a part of a native NPV-alpha baculovirus clade Ia genome to reconstitute a genome that is capable of forming an infectious nucleopolyhedrovirus. This can be achieved by using advanced recombination technologies such as ET recombination (in vitro and in vivo) (Zhang, Y, et al., (1998) Nature Genet., 20, 123-128, Hill, F. et al., (2000) Genomics, 64, 111-113.) to assemble these synthetic DNAs into the part of a native NPV-alpha baculovirus genome to yield functional virus.

In a sixth aspect the present invention provides a method for producing a NPV alpha baculovirus clade Ia virus by introducing a genome of the first or second aspect or a genome producible according to the method of the fifth aspect of the invention into a cell, preferably one of the cells indicated above regarding the fourth aspect

### BRIEF DESCRIPTION OF THE DRAWINGS

FIG. 1: Blueprint of the Baculovirus Genome. The annotated genome of Autographa californica multicapsid nuclear polyhedrosis virus (AcMNPV) is shown in a schematic representation (top). Genes are scored (bottom) on a scale from essential genes that are conserved (no deletion category, shades of green color to black) to non-essential genes (shown to be possibly deleteable, shades of red color). The classification applied is detailed in the legend (bottom). The annotated genome map was generated by a self-developed Perl program. Essential and non-essential genes are not randomly distributed but cluster in the genome. The upper semicircle is composed to 56% of the essential genes (and 44% of non-essential genes), the lower, more conserved semicircle is composed to 71% of essential genes (and 29% of non-essential genes).

FIG. 2: Shows an alignment of homologs of a protein tyrosine phosphatase present in NPV alpha baculovirus clade Ia viruses from 11 different NPV alpha baculovirus clade Ia viruses. To compile the proteins for this alignment the amino acid sequence of the protein tyrosine phosphatase AcMNPV according to SEQ ID NO: 13 was used in a PBLAST scarch of non-redundant protein sequences. The amino acid sequences of homologs of the protein tyrosine phosphatase from other NPV alpha baculovirus clade Ia viruses identified, comprised the following: a homolog from RoMNPV (SEQ ID NO: 14), a homolog from BmNPV (SEQ ID NO: 15), a homolog from MaviMNPV (SEQ ID NO: 16), a homolog from CfDefMNPV (SEQ ID NO: 17), a homolog of AgMNPV (SEQ ID NO: 18), a homolog from EppoNPV (SEQ ID NO: 19), a homolog from AnpeNPV (SEQ ID NO: 20), a homolog from CfMNPV (SEQ ID NO: 21), a homolog from OpMNPV (SEQ ID NO: 22), and a homolog from HycuNPV (SEQ ID NO: 23). Using such an alignment the position of the ptp gene in the genome of every NPV alpha baculovirus clade Ia virus can be determined and subsequently deleted.

FIG. 3: hr4 and hr5 containing region (bp 99.182 to 121.072 of the baculoviral genome). The original (wild-type) genome region is shown, with essential (black arrows) and non-essential (chequered arrows) gene regions high-lighted. Genes are annotated and hr regions indicated.

FIG. 4: Minimized hr4 and hr5 containing region. The synthetic designed DNA piece to replace the original DNA segment is shown, with deleted gene loci and maintained essential genes. A YFP expression cassette is introduced, as well as an antibiotic marker (Amp) flanked by two sites for homologous recombination.

FIG. 5: Experimental procedure of genome grafting for replacing the wild-type sequence with the designer DNA. First, the wild-type genomic region is eliminated by homologous recombination and replaced by a gentamycin marker (left and middle). Next, the synthetic designer DNA is assembled by biobrick method from three synthetic precursor DNAs (bottom right). By a second homologous recombination step, the synthetic DNA is introduces into the wild-type genome, replacing the gentamycin marker (left, bottom) giving rise to a hybrid genome that is partly wild-type and partly synthetic (SynBac1.0) as proof-of-concept (PoC).

FIG. 6: Genome analysis of SynBac1.0. The partly synthetic baculovirus genome containing the rewired designer DNA region was analyzed on the DNA level by analytical PCR, showing that the genes that were eliminated are indeed absent. The agarose gel on the left shows PCR amplification of selected genes of the wild-type BV genome, and the agarose gel on the right the same for the SynBac1.0 genome. The selection of genes represents a sampling of PCR amplified DNA, the absence or presence of which in the PCR experiment unambiguously shows that the original DNA is replaced by the synthetic DNA.

FIG. 7: Analysis of live SynBac1.0. A YFP fluorescence emission spectrum of the YFP expression in insect cells infected with serially passaged SynBac1.0 virus. The X-axis shows the wavelength (lambda) of the emission signal, and the Y-axis shows the fluorescence intensity in arbitrary units. B Coomassic Blue staining of the protein content of the infected cells. 1: Cell control, 2: Marker, 3 and 4: SyBac1.0, 5: Virus control. p10 (10 kDa protein visible at the bottom of the gel as a strong band), which is strongly expressed in the wild-type virus control infected cells, is not present in SynBac1.0 infected cells.

### EXAMPLES

The Examples are designed in order to further illustrate the present invention and serve the purpose to allow a better understanding of the invention. They are not to be construed as limiting the scope of the invention in any way.

### Example 1

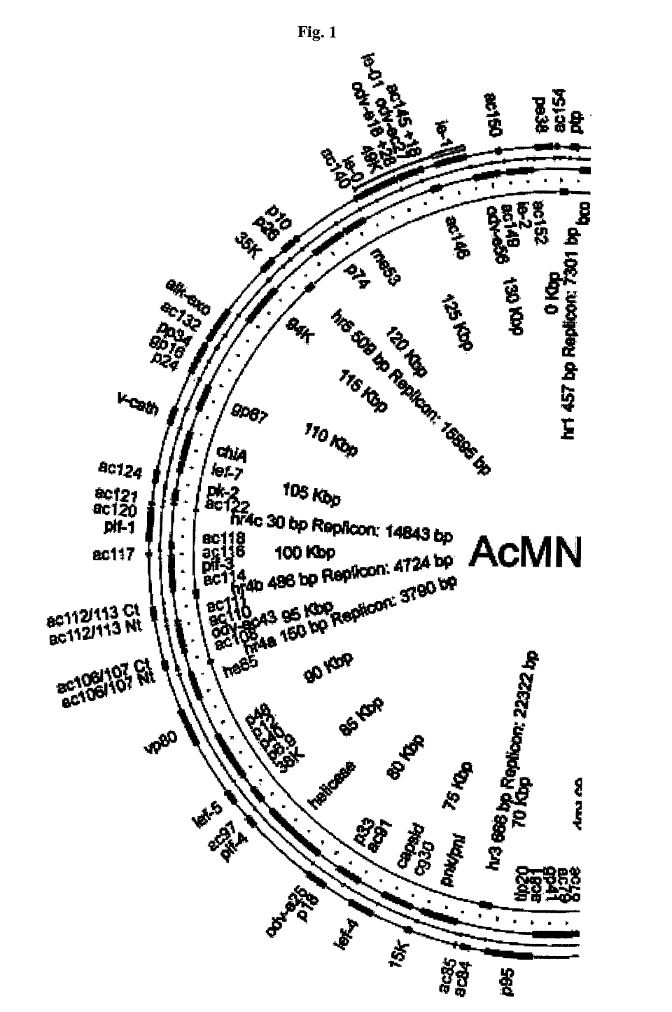

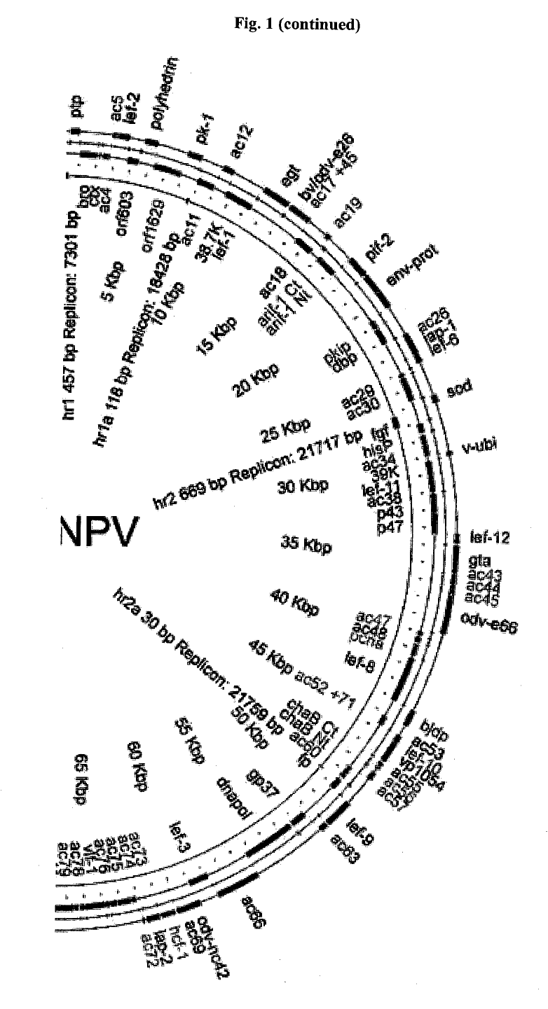

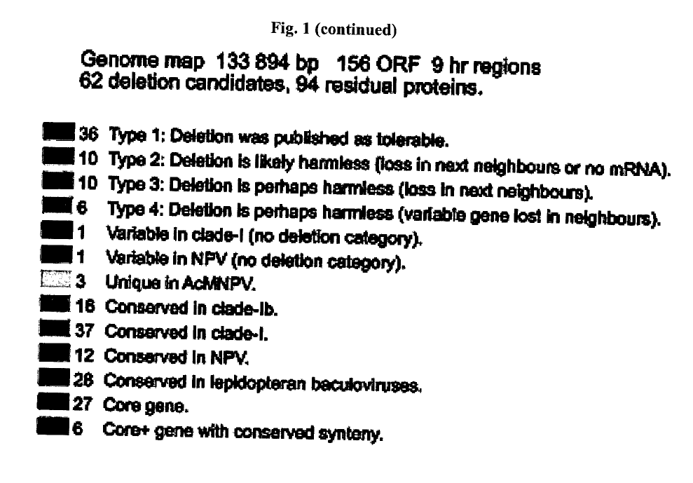

The data used for creating the baculovirus genome map shown in FIG. 1 were derived from mining 1253 relevant papers found in NCBI PubMed and published up to October 2011 on information for e.g. genome sequences, gene essentiality and conservation, protein product function and localization, protein-protein interactions, mRNA expression and gene regulation.

All available database annotations on gene product function were collected from NCBI Genebank, NCBI Protein, NCBI VOG clusters of related viral proteins and the UniProt database by Perl programs. Additionally 53 baculovirus genomes available in October 2011 in the NCBI ReSeq nucleotide database were analyzed for orthologous protein genes by clustering protein sequences downloaded from the NCBI protein database with a Perl program, which piped the clustering program blastclust, a part of the NCBI C-toolkit legacy blast package (Vijayachandran LS, Thimiri Govinda Raj D B, et al (2013) Bioengineered 4:5, 1-9). Categories for conservation/variability were assigned for following different lineages of baculoviruses: all baculoviruses with conserved synteny (core+), all baculoviruses (core), lepidopteran baculoviruses, NPV (alphabaculoviruses), NPV clade I, NPV clade Ia, variable for next neighbours of AcMNPV, unique for AcMNPV). For gene essentiality classification the grades of gene conservation were integrated with published information on mRNA expression (no mRNA observed), expression in different developmental stages (immediate early, early, late, very late), content of stage-specific promoter motifs in the upstream regions and gene product function and localization indicating non-essentiality for virus propagation in cell culture (e.g. host interaction factors, oral infectivity factor, occlusion-derived virus or occlusion-body localized, other auxiliary proteins, some genes acquired from the host genome). Protein genes, which were already published as non-essential, were categorized as type 1 deletion category (deletion is harmless), that having no known mRNA expression or have been proven as non-essential in the next relatives of AcMNPV (like BmNPV) as type 2 (deletion is likely harmless), that having presumably non-essential functions and are variable in alphabaculoviruses or such protein genes having unknown functions and are variable in next neighbours as type 3 (deletion perhaps harmless) and that with suspect non-essential functions and variability in other Alphabaculovirus genomes but conservation in next relatives as type 4 (deletion perhaps harmless). The genome map was drawn by designing a Perl program, which imports a table of the categorized gene data and exports an image made by the Perl packages GD and GD::Simple.

### Example 2

According to Example 1, the inventors identified, by comparative genomic analyses and data mining, regions in the baculovirus genome that can be rewired advantageously to generate an improved baculovirus for drug discovery purposes.

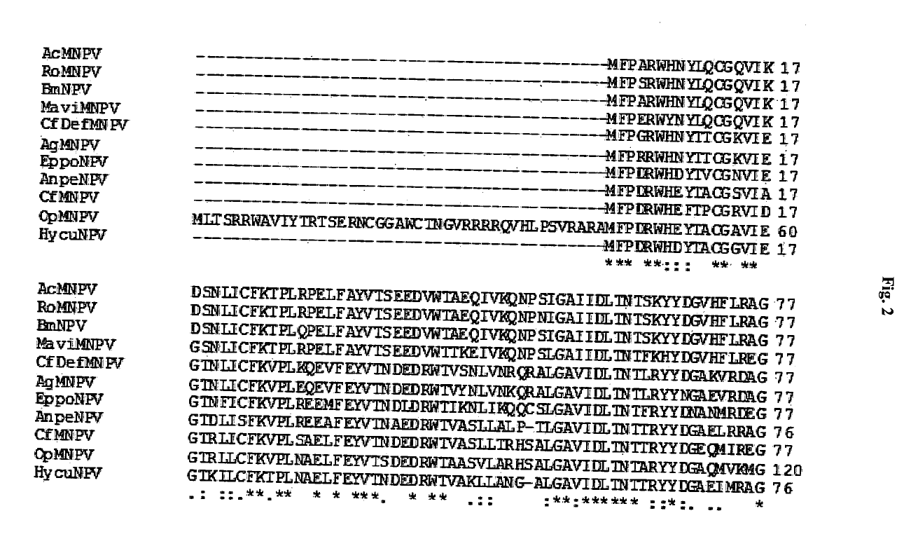

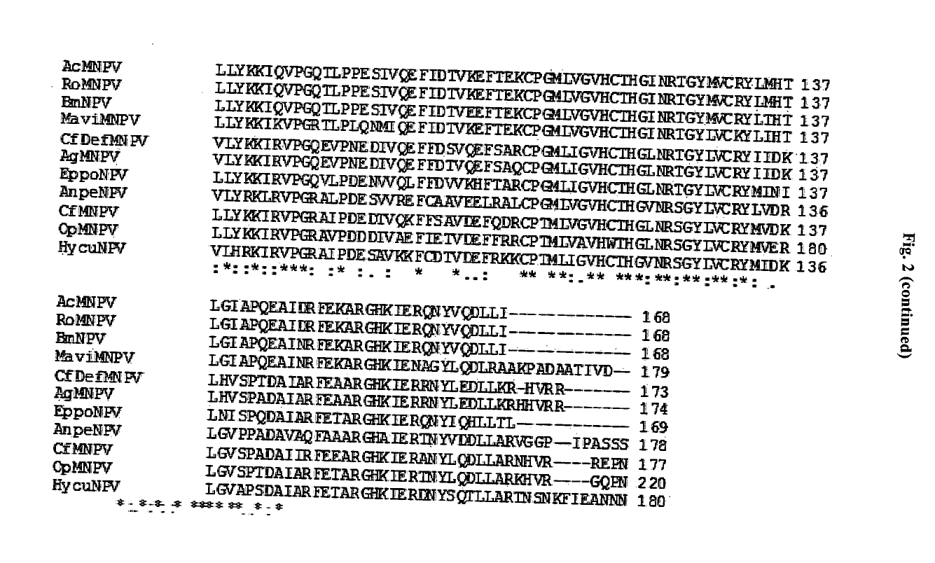

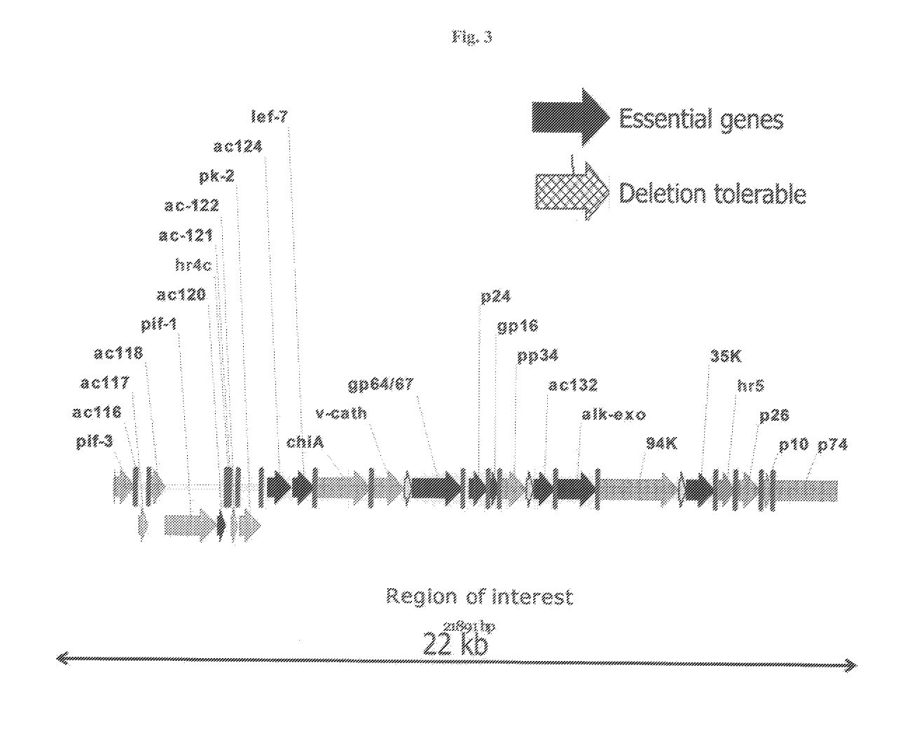

For a proof-of-concept of this approach, the hr4 and hr5 containing region was chosen. This region is located between bp 99.182 and 121.072 on the baculoviral genome, and contains in addition to the hr4, hr5 regions 22 genes of which, based on the inventors' studies, 12 are non-essential and 10 are essential. In terms of size, this 22 kb segment contains less than 10 kb of essential DNA material including genes, promoters and terminators, and around 20 kb of DNA with functions the deletions of which can be tolerated or even be enhancing the performance of the virus in cell culture. The hr4, hr5 regions and their essential and non-essential portions are shown in FIG. 3.

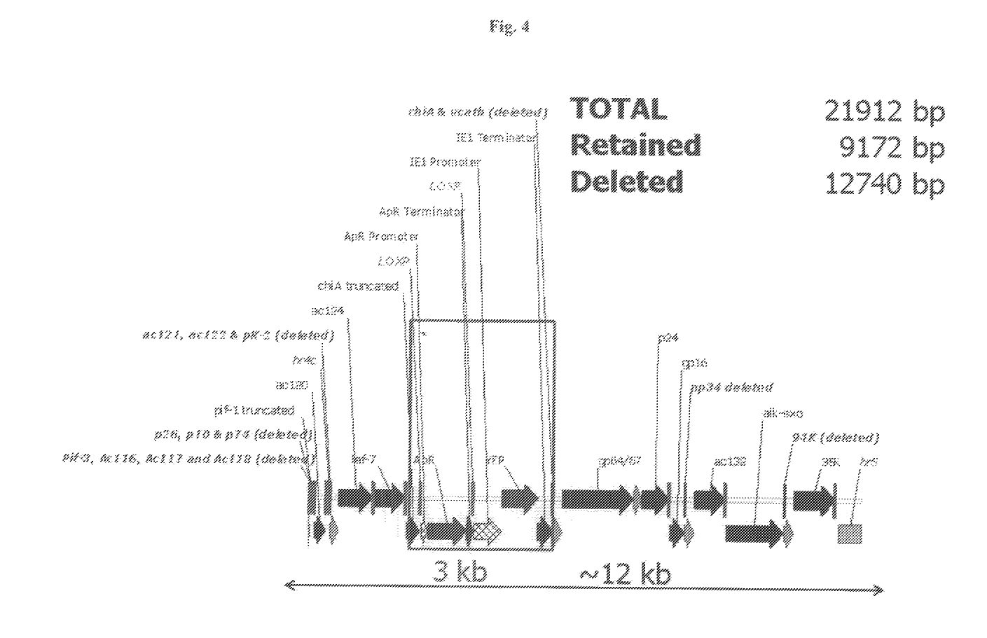

The inventors designed a synthetic DNA corresponding to a rewired and minimized version of this segment (see FIG. 4). They created this piece of DNA from custom synthesized DNA pieces by applying BioBrick methods (see Sleight C. S. et al Nucleic Acids Res. 2010).

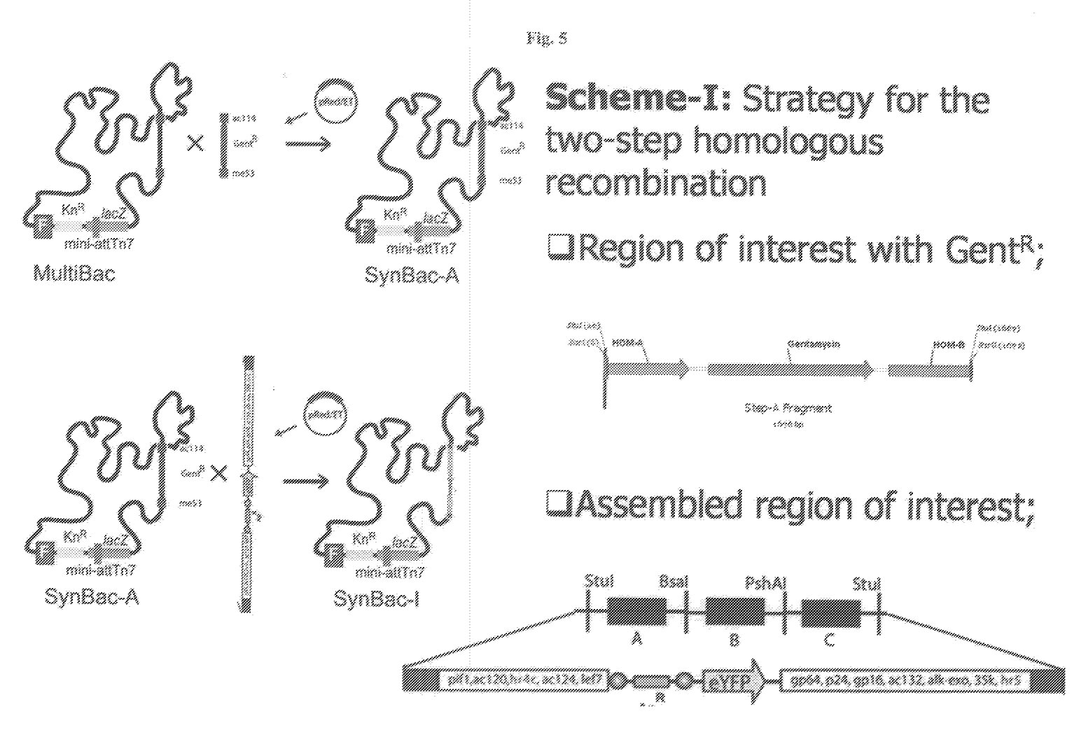

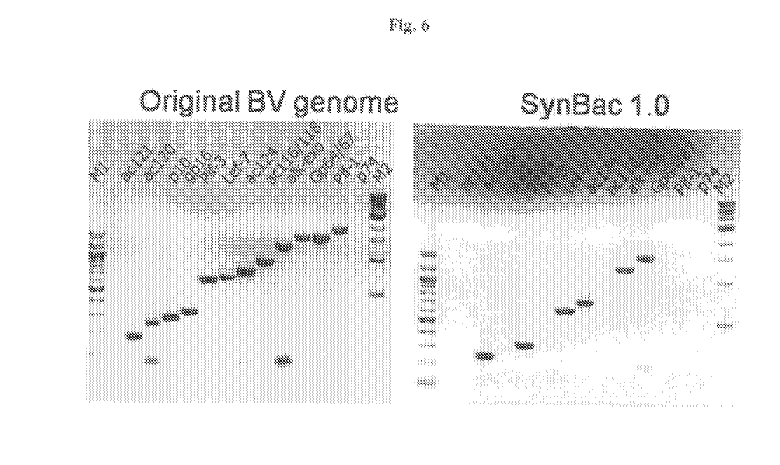

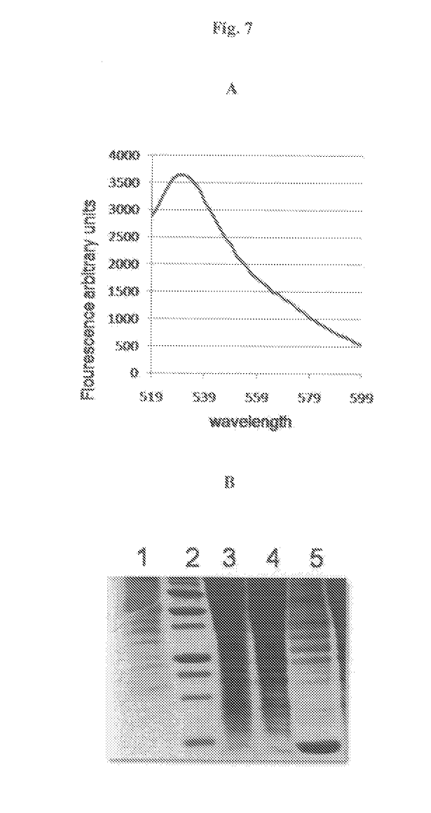

Next, they used homologous recombination techniques (see Zhang Y et al Nat. Biotechnology (2000) to graft this synthetic DNA into the wild-type baculoviral genome, replacing the original wild-type sequence in this segment with the designer DNA (see FIG. 5). The partly synthetic baculovirus genome containing the rewired designer DNA region was analyzed on the DNA level by analytical PCR, showing that the genes that were eliminated are indeed absent (see FIG. 6). To assess viability of the virus, insect cells were infected with serially passaged Synbac1.0 virus and the expression of YFP from the viral backbone was measured (see FIG. 7A). This demonstrates that SynBac1.0 is infectious and viable. Also, the protein content of the infected cells was examined with respect to exemplary proteins. p10 (10kD mass, shown as an representative example) was clearly absent in the infected cells, although this protein is strongly expressed protein in the wild-type baculovirus (see FIG. 7 B).

Thus, the resulting hybrid (i.e. partly synthetic, partly wild-type) baculoviral genome (SynBac1.0) proved to be fully functional, infectious and able to produce heterologous protein. This shows that the approach of the invention can be reduced to practice.

1. An Autographa californica multicapsid nucleopolyhedrovirus (AcMNPV) genome, wherein the number of base pairs is reduced in comparison to a native AcMNPV genome by at least 25.7% and which assembles into an infectious baculovirus.

2. The AcMNPV genome according to claim 1 lacking at least one gene selected from one of the following groups consisting of:
ac85, ac116, cg30, hcf-1, ac56, and pcna;
ac44, ac57, ac84, ac112/113 Ct, ac112/113 Nt, ac118, ac122, ac152, ie-01 and hispP;
ac11, ac30, eta, ac63, 15k, ac97, ac121, ac140, ac146 and ac149; and
odv-e66, p43, odv-nc42 or odv-e56, ptp, bro, ctx, orf603, polyhedrin, egt, bv/odv-e26, ac18, pif-2, env-prot, iap-1, sod, fgf, vubi, gp37, ac69, iap-2, pnk/pn1, ac91, odv-e28 pif-4, pif-3, pif-1, pk-2, chiA, v-cath, pp34, 94K, p26, p10, p74, ac145, and ac150.

3-5. (canceled)

6. The AcMNPV genome according to claim 1 lacking at least one gene selected from the group consisting of pif-1, p26, p10, p74, pif-3, ac116, ac117, ac118, ac121, ac122, pk-2, chiA, v-cath, pp34 and 94K, wherein pif-1 and chiA are partially deleted such that the promoter regions of the adjacent genes remain.

7. An Autographa californica multicapsid nucleopolyhedrovirus (AcMNPV) genome, lacking at least one gene selected from the group consisting of pif-1, p26, p10, p74, pif-3, ac116, ac117, ac118, ac121, ac122, pk-2, chiA, v-cath, pp34 and 94K, of which pif-1 and chiA may only be partially deleted such that the promoter regions of the adjacent genes remain, and which assembles into an infectious baculovirus.

8. The AcMNPV genome according to claim 1, wherein the genome is further lacking the 5′-UTR and/or 3′-UTR of the genes, the spacers 5′ and/or 3′ of the genes, and/or one or more of the heterologous repeat sequences.

9-10. (canceled)

11. The AcMNPV genome according to claim 1 comprising at least one of the following:
genes encoding helicase, 38K, lef-5, 49K and odv-e18+28;
genes encoding lef-2, lef-1, p47, lef-8 vp1054, lef-9, dnapo1, ac66, vlf-1, gp41, ac81, p95, capsid, lef-4, P33, p18, odv-e25, p6.9, odv-ec43, alk-exo, and odv-ec27;
genes encoding pk-1, 38.7K, dbp, lef-6, ac29, 39K, lef-11, ac38, ac53, fp, lef-3, ac75, ac76, ac78 tlp20, p40, p12, p48, ac106/107 Nt, ac106/107 Ct, ac110, me53 and ie-1:
genes encoding ac12, ac34, ac55, and ac108;
genes encoding ac4, ac5, orf1629, ac17+45, ac19, arif-1 Ct, arif-1 Nt, pkip, ac26, lef-12, ac43, ac48, bidp, ac72, ac73, ac74, ac79, ac11, ac114, ac120, ac124, lef-7, gp67, gp16, ac132, ie-2, pe38; and
genes encoding ac45, ac47, ac52+71, he65, 35K and ac154;

12-16. (canceled)

17. The AcMNPV genome according to claim 1, comprising at least one of the genes of the group consisting of ac120, ac124, lef-7, gp64/67, p24, gp16, ac132, alk-exo, and 35K.

18. A Bombyx mori nucleopolyhedrovirus (BmNPV) genome, wherein the number of base pairs is reduced in comparison to a native BmNPV genome by at least 18.31% and which assembles into an infectious baculovirus.

19. The BmNPV genome according to claim 18 lacking at least one gene selected from the group consisting of bm45 ac56, cg30, and bm95a ac116.

20. The BmNPV genome according to claim 18 lacking at least one gene selected from the group consisting of bm35 ac44, odv-e66, bm46 ac57, and bm99 ac122.

21. The BmNPV genome according to claim 18 lacking at least one gene selected from the group consisting of bm4 ac11, bm51 ac63, 15K, bm98a ac121, bm122 ac146, and bm125 ac149.

22. The BmNPV genome according to claim 18 lacking at least one gene selected from the group consisting of polyhedrin, egt, bv/odv-e26, bm10 ac18, pif-2, env-prot, iap-1, fgf, v-ubi, bm57 ac69, bm74 ac91, pif-3, pif-1, chi-a, v-cath, pp34, bm110a 94K ac134, p26, p10, p74, bm121/ac145, bm126 ac150, ptp, bro-d, gta, and gp37.

23. The BmNPV genome according to claim 18 further lacking the 5′-UTR and/or 3′-UTR of the genes, lacking the spacers 5′ and/or 3′ of the genes, and/or lacking one or more of the heterologous repeat sequences.

24-25. (canceled)

26. A AcMNPV genome according to claim 1 or a BmNPV genome according to claim 18 further comprising a nucleotide sequence heterologous to the AvMNPV or BmNPV genome.

27. The AvMNPV genome according to claim 26, wherein the heterologous nucleotide sequence comprises a Tn7 site, a nucleotide sequence encoding a resistance gene, a Homing endonuclease site, a mutated fp gene, loxP sites, IE1/polyhedrin/p10 promoter, and/or a nucleotide sequence encoding a fluorescent protein.

28. An infectious AvMNPV virus comprising a genome according to claim 1.

29. An infectious BmNPV virus comprising a genome according to claim 18.

30. A cell infected with a virus according to claim 28 or 29.

31. The cell according to claim 27, wherein the cell is selected from the group consisting of Ao/I, Hi5, Sf9, Sf21, Ao38, Drosophila S2, T.ni, FTRS-AoL1/-AoL2/-AfL, BCIRL/AMCY-AiOV-CLG, BCIRL/AMCY-AiTS-CLG, HCRL-ATO10/ATO20, BCIRL/AMCY-AfOV-CLG, BCIRL/AMCY-AfTS-CLG, RML-2, NISES-AnPe-426, NISES-Anya-0611, BCIRL/AMCY-AgE-CLG-1/2/3, UFL-AG-286, FTRS-AbL81, SES-Bma-O1A/R, Bm-N/-5/-21E-HNU5, NIV-BM-1296/-197, SES-Bm-130A/30R/e 21A/e 21B/e 21R, SES-BoMo-15A/-C129/-JI25, SPC-Bm36/-Bm40, WIV-BS-481/484, FPMI-CF-1/2/3, FPMI-CF-203, FPMI-CF-50/60/70, IPRI-CF-1/10/12, IPRI-Cf124, IPRI-CF-16/-16T, IPRI-CF-5/-6/-8, CP-1268, CP-169, CpDW1-15, IZD-Cp 4/13, IZD-CP1508/-CP2202/-CP2507/-CP0508, SIE-EO-801/-803, IPLB-Ekx4T/-Ekx4V, EA1174A, EA1174H, IAFEs-1, BCIRL-HA-AM, CSIRO-BCIRL-HA1-3, CSIRO-BCIRL-HP 1-5, BCIRL/AMCY-HzE-CLG1-9, BCIRL-HZ-AM1-3, IMC-HZ-1, IPLB-HZ-1074-5, IPLB-HZ-1079, IPLB-HZ-110, IPLB-HZ-124Q, BCIRL/AMCY-HvE-CLG1-3, BCIRL/AMCY-HvOV-CLG, BCIRL/AMCY-Hv-TS-GES, BCIRL-HV-AM1-2, IPLB-HvE1a/-It, IPLB-HvE1s, IPLB-HvE6a/-It, IPLB-HvE6s/-It, IPLB-HvT1, FTRS-HmL45, FTRS-HIL1-2, NIAS-LeSe-11 IPLB-LD-64-67, IPLB-LdEG/-LdEI/-LdEIt/-LdEp/-LdFB, IZD-LD1307/-LD1407, UMN-MDH-1, HPB-MB, IZD-MB0503/MB0504/MB1203/MB2006/2007/2506, MB-H260, MbL-3, NIAS-MaBr-85/92/93, NIAS-MaBr-92, NIAS-MB-19/25/32, SES-MaBr-1/2/3/4/5, FPMI-MS-12/4/5/7, MRRL-CH-1/2, BPMNU-MyCo-1, IPLB-O1E505A/s, IPLB-O1E7, IPRI-OL-12/13/4/9, BCIRL/AMCY-OnFB-GES1/2, UMC-OnE, FTRS-PhL, Px-58/-64, ORS-Pop-93/-95, BTI-PRIOB/-PR8A1/-PR8A2/-PR9A, NIAS-PRC-819A/-819B/-819C, NYAES-PR4A, IAL-PID2, IPLB-PiE, UMN-PIE-1181, BCIRL/AMCY-PxLP-CLG, IPLB-PxE1/-PxE2, PX-1187, BCIRL-PX2-HNU3, BTI-Pu-2, BTI-Pu-A7/-A7S, BTI-Pu-B9, BTI-Pu-M, BTI-Pu-MIB, FRI-SpIm-1229, BCIRL/AMCY-SeE-CLG/-CLG4/-CLG5, Se3FH, Se4FH, Se5FH, Se6FHA, Se6FHB, SeHe920-1a, UCR-SE-1, BCIRL/AMCY-SfTS-GES, IAL-SFD1, Sf1254, IPLB-Sf21AE, HPB-SL, SPC-SI-48/-52, UIV-SL-373/-573/-673, IBL-SLIA, NIV-SU-893/-992, BCIRL-503-HNU1/504-HNU4, BCIRL/AMCY-TnE-CLG 1/-TnE-CLG1MK, BCIRL/AMCY-TnE-CLG2/-TnE-LG2MK/-TnE-CLG3/-TnTS-GE3/-TnTS-GES3, BTI-TN5B1-4/-TN5C1/-TN5F2/-TN5G2A1/BTI-TN5G3/-TN5G33, IAL-TND1, IPLB-TN-R, and TN-368.

32. A method for producing an AvMNPV genome according to claim 1, or a BmNPV genome according to claim 15 or a AvMNPV or BmNPV genome according to claim 26 comprising the step of chemically synthesizing all or part of the genome.

33. The method according to claim 32, wherein the part of the genome flanks the regions that are deleted from the genome.

34. The method according to claim 32, wherein the part of the genome is inserted into a part of a native genome to reconstitute a genome that is capable of forming an infectious nucleopolyhedrovirus.

35. A method for producing a AcMNPV or BmNPV virus by introducing a genome of claim 1 or claim 18, respectively, or chemically synthesizing all or part of the genome, into a cell.

36. The BmNPV genome according to claim 26, wherein the heterologous nucleotide sequence comprises a Tn7 site, a nucleotide sequence encoding a resistance gene, a Homing endonuclease site, a mutated fp gene, loxP sites, IE1/polyhedrin/p10 promoter, and/or a nucleotide sequence encoding a fluorescent protein.
